(19) United States
(12) Patent Application Publication
(10) Pub. No.: US 2003/0138430 A1
(43) Pub. Date: Jul. 24, 2003

(54) Pharmaceutical comprising an agent that blocks the cell cycle and an antibody

(76) Inventors: Julie Beth Stimmel, Research Triangle park, NC (US); Linda Margarite Thurmond, Research Triangle Park, NC (US); Vincent Clark Knick, Research Triangle Park, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/239,278

(22) PCT Filed: Mar. 22, 2001
(86) PCT No.: PCT/US01/09368

(51) Int. Cl. A61K 51/00; A61K 39/395; A61K 33/24; A61K 31/7072; A61K 31/704; A61K 31/337; A61K 31/4745

(52) U.S. Cl. 424/155.1; 424/649; 514/34; 514/102; 514/449; 514/283; 514/49; 424/1.49

(57) ABSTRACT

Pharmaceutical combinations comprising an agent that arrests target cells in the G₂ and/or M phase of the cell cycle and another therapeutic agent that targets an internalising cell surface structure such as an antigen. Use in the manufacture of a medicament and in methods of medical treatment, particularly in the treatment of diseases of cell cycle regulation such as cancer are disclosed.

### FIELD OF THE INVENTION

The present invention relates to pharmaceutical combinations, that is combinations of therapeutically active agents in the treatment of mammalian patients particularly those afflicted with a disease of cell cycle regulation such as cancer or a disease or disorder of metabolic dysfunction and methods of medical treatment comprising the same. The present invention more particularly concerns the combined use of an agent that is capable of affecting cell growth (i.e. number) by blocking (or retarding) progression of the cell cycle in G₂ and/or M (herein “G₂/M agents”) and another therapeutic agent. Pharmaceutical preparations comprising a G₂/M agent and another therapeutic agent whose therapeutic effectiveness depends at least in part on the presence of a cell surface structure on the target cell that recycles through are also disclosed. Other aspects, objects and advantages of the present invention will be apparent from the description below.

### BACKGROUND OF THE INVENTION

The cell cycle refers to a sequence of events between one mitotic division and another in a cell. A quiescent resting phase (G₀) is followed by a growth phase (G₁), then by DNA synthesis phase (S). A second growth phase of cell enlargement (G₂) and DNA replication (M phase) is followed by division of the cell into two progeny cells. DNA is stained with intercalating dyes (i.e. propidium iodide or 4′,6′-diamidino-2-phenylindole (DAPI)) and using flow cytometry, the cellular amount of the DNA can be used to determine the cell cycle distribution. Interference with cellular machinery may inhibit progression through the cell cycle. For example, specific chemotherapeutic agents may block progression in either G₂ and/or M. In other words exposure to certain drugs, e.g. chemotherapeutic agents will for example arrest individual cells in G₂ and/or M until eventually most, or all of the cells in a population cease progression through the cell cycle and arrest in G₂ and/or M. While a few cell surface structures such as proteins have been identified as produced solely at certain phases of the cell cycle, and therefore can serve as markers of cell cycle status, most others are produced across the cell cycle but at higher or lower levels at certain points.

Variation of antigen density across the cell cycle is typical for sacroma antigens p102 and p200 (Song S, Anticancer Research 16(3A): 1171-5 (1996)), the leukaemia/lymphoma-associated antigen JD118 (Czuczman et al; Cancer immunology, immunotherapy 36(6):387-96 (1993)) and the gastric tumour antigen PC1 (Wei et al., J. Oncology 9(3): 179-182 (1987)). A few tumour antigens have been reported to be cell-cycle independent, e.g. liver metastates 3H4 (Wulf et al., J Cancer research and clinical oncology 122(8): 476-82, 1996) and small cell lung cancer antigens (Fargion et al., Cancer Research 46:2633-2638 (1986)). See also Crissman et al; 1990. Cytochemical techniques for multivariate analysis of DNA and other cell constituents, In Flow Cytometry and Cell sorting, 2^{nd} edtn, pp227-247, Wiley-Liss, New York.

A process by which the cell's plasma membrane including associated structures (e.g. proteins, glycoproteins) invaginate is endocytosis. Through endocytosis, the membrane and associated structures are taken up within the cell and subject to further processing by cellular machinery. It has been shown that receptor endocytosis is required for agonist-induced mitogenic signalling of various tyrosine kinase growth receptors such as receptors for epidermal growth factor receptor (Vieira, A V, Lamaze, C., and Schmid, S L (1996) Nature 274, 2086-2089), nerve growth factor receptor (Riccio, A, Pierchala, B A, Ciarallo, C L, and Ginty D D (1997) Science 277, 1097-1100), and insulin growth factor receptor 1 (Chow J C, Condorelli G, and Smith R J (1998) J Biol. Chem. 273, 4672-4680), as well as G protein-coupled receptors, such as endothelial cell-derived G protein-coupled receptor (EDG-1) and chemokine receptor CXCR1 (Barlic J, Khandaker, M H, Mahon, E, Andrews, J, DeVries, M E, Mitchell, G B, Rahimpour, R, Tan, C M, Ferguson, S S G, and Kelvin D J (1999) J Biol. Chem. 274 (23), 16287-16294). In addition, endocytosis has been implicated in signalling events involved in integrin activation. Integrins link extracellular matrix proteins to cytoskeletal proteins and actin filaments on the cytoplasmic face and have been shown to regulate agonist-induced protein phosphorylation (Clark, E A, Shattil, S J and Brugge, J S (1994) Trends Biochem. Sci. 19, 464).

Classical markers of receptor-mediated endocytosis are macromolecules such as transferrin, low-density lipoprotein or asialoglycoprotein receptors. These macromolecules bound to specific receptors at the cell surface are internalised by the cell via endocytosis. Initially, macromolecules are internalised into early endosomes and once there, are either recycled to the plasma membrane or became concentrated with sorting endosomes before being routed towards lysosomes. Microtubule-dependent transport is an integral component of many of the membrane-trafficking events involved in endocytosis, secretion, transcytosis, and membrane organisation and maintenance (Cole, N. B. and Lippincott-Schwartz, J. (1995) “Organisation of organelles and membrane traffic by Microtubules” Curr. Opin. Cell Biol. 7, 55-64; Goodson, H. V., Valetti, C., and Kreis, T. E. (1997) “Motors and membrane traffic” Curr. Opin. Cell Biol. 9, 18-28.). Numerous studies support a role for cytoplasmic dynein-driven vesicle transport in movement form the early endosomes to late endosomes and/or lysosomes (Aniento, F., Emans, N., Griffiths, G., and Gruenberg, J. (1993) “Cytoplasmic dynein-dependent vesicular transport from early to late endosomes” J. Cell Biol. 123, 1373-1387; Novikoff, P. M., Cammer, M., Tao, L, Oda, H., Stockert, R. J., Wolkoff, A. W., and Satir, P. “Three-dimensional organisation of rat hepatocyte cytoskeleton: relation to the. asialoglycoprotein endocytosis pathway” J. Cell Sci. 109, 21-32; Oda, H., Stockert, R. J., Collins, C., Yoon, Y., and Jung, M. K. (1990) “Interaction of the micotubule cytoskeleton with endocytic vesicles and cytoplasmic dynein in cultured rat hepatocytes” J. Biol. Chem. 270, 15242-15249). Cultured cells have demonstrated that changes in the microtubule array can retard the movement transferrin receptor and epidermal growth factor receptor from the plasma membrane to early endosomes (Jin M., and Snider M. D. (1993) “Role of microtubules in transferrin receptor transport from the cell surface to endosomes and the Golgi complex” J. Biol. Chem. 268, 18390-18397; Thatte, H. S., Bridges, K. R., and Golan D. E. (1994) “Microtubule inhibitors diffentially affect translational movement, cell surface expression and endocytosis of transferrin receptors in K562 cells” J. Cell Physiol. 160, 345-357; Van't Hof, Ob J., Defize, L. H. K., Nuijdens, R., De Brabander, M., Verkleij, A. J., and Boonstra, J. (1989) “Dynamics of epidermal growth factor receptor internalisation studied by nanovid light microscopy and electron microscopy in combination with immunogold labeling” Eur. J. Cell Biol. 48, 5-13), but the mechanism by which this occurs is not known. However, it is clear that microtubules play a central role in the movement of internalised membrane-bound material through the endosomal and degradative compartments.

The conventional therapeutic approaches to the treatment of cancer include surgery, radiotherapy and chemotherapy in various combinations; however, response rates for some types of cancer have not improved significantly in the last 20 years. The major limitation of chemotherapy and radiotherapy is the non-selective targeting of both normal and tumour cells that result in toxic side effects. In the search for less toxic and more specific treatment alternatives, various types of immunotherapy have been investigated. Among these modalities, strategies based on monoclonal antibodies have been applied to a broad spectrum of malignancies. The utility of monoclonal antibodies is based upon their clonal antigen specificity, i.e. molecular recognition of specific epitopes which may comprise an antigen and to bind to these antigens with high affinity. Monoclonal antibodies can bind to antigens expressed uniquely or preferentially on the surface of malignant cells and hence can be used to specifically target and destroy tumour cells. Antibodies may be constructed as delivery vehicles for drugs or DNA or as conjugates with radionuclides. Binding of naked antibody to target cells may also activate innate antitumour immune functions such as antibody-dependent cell-mediated cytotoxicity (ADCC) and complement mediated cytotoxicity (CMC), either of which may result in lysis or phagocytosis of the targeted cell. Both ADCC and CMC are antibody-dose related immune functions and it is therefore desirable to get as much antibody bound to target cells as possible. One way of achieving this objective is to increase the amount of antigen expressed on the cell surface which may effectively increase antibody functions such as, for example, ADCC of the target cells by virtue of getting more antibody bound to cells.

Increased cell surface expression of some pancreatic tumor antigens (Mukerjee, S., McKnight, M. E., Nasoff, M., and Glassy, M. C. “Co-expression of tumor antigens and their modulation by pleiotrophic modifiers enhance targeting of human monoclonals antibodies to pancreatic carcinoma” Human Antibodies 9, 9-22 (1999)) and epidermal growth factor receptor (Zuckier G. and Tritton T. R. “Adriamycin Causes Up Regulation of Epidermal Growth Factor Receptors in Actively Growing Cells” Experimental Cell Research 148, 155-161 (1983); Hanauske A.-R., Depenbrock, H., Shirvani, D., and Rastetter J. “Effects of Microtubule-disturbing Agents Docetaxel (Taxotere®), Vinblastine and Vincristine on Epidermal Growth Factor-receptor Binding of Human Breast Cancer Cell Lines In Vitro” Eur. J. of Cancer, 30A (11), 1688-1694 (1994); Depenbrock, H., Shirvani, A., Rastetter J., and Hanauske, A.-R. “Effects of vinorelbine on epidermal growth factor-receptor binding of human breast cancer cell lines in vitro” Invest. New Drugs 13, 187-193 (1995)) following pre-treatment with G₂/M agents and agents that target microtubules has been reported. However, the scope and mechanism of these reported increases in surface antigen is unclear. In the study by Mukerjee et al., the authors claimed that three unique cell surface structures on a pancreatic cell line had higher co-expression levels relative to untreated controls following exposure to interferons-α, -β, and -γ, or the microtubule-targeting agents vinblastine, colchicine, and vincristine. This study was accomplished only on the PANC-1 cell line, and was not common to adenocarcinomas; furthermore, while a greater percentage of cells co-expressed antigen in some cases, an increase in antigen density per cell was not demonstrated. The authors speculate that this approach to therapy might work due to enhanced antigen turnover, but did not characterise whether or not these cell surface structures are internalised. Lastly, the effect of interferons on at least one ganglioside antigen suggests that the mechanism of increased in surface density may be the result of increased gene expression. In a series of studies from Hanauske's lab on another surface antigen, the effect of a variety of cytotoxic agents on the binding of epidermal growth factor (EGF) to its receptor was evaluated in adenocarcinoma cell lines in culture. In an early study, doxorubicin increased EGF binding, though vinblastine and cisplatin caused a reduction in the binding affinity. In two later studies, the microtubule-targeting agents vincristine, vinblastine, docetaxel, and vinorelbine (Navelbine) caused an increase in EGF binding. The authors concluded that this was due to an increase in the number of binding sites. These studies were conducted in consideration of the natural ligand as a mitogenic peptide, and were not therapy-directed. Zuckier and Tritton also demonstrated increased EGF binding (binding of the natural ligand) following treatment with doxorubicin and obtained similar conclusions about an increase in the number of EGF binding sites. The authors did not demonstrate that this increase in the numbers of receptors provided a therapeutic benefit, and the distinction between the binding of a natural ligand and a therapeutic monoclonal antibody was not drawn. Successful antibody combinations that target epidermal growth factor receptor include the G₂/M agents doxorubicin and cisplatin (Baselga, J., Norton, L., Masui, H., Pandiella, A., Coplan, K., Miller Jr., W., and Mendelsohn, J. “Antitumor Effects of Doxorubicin in Combination with Anti-epidermal Growth Factor Receptor Monoclonal Antibodies” J. Natl. Cancer Inst. 85 (16) 1327-1333 (1993); Fan, Z., Baselga, J., Masui, H., and Mendelsohn, J. “Antitumor Effect of Anti-epidermal Growth Factor Receptor Monoclonal Antibodies plus cis-Diaminedichloroplatinum on Well Established A431 Xenografts” Cancer Research 53, 4637-4642 (1993)). Doxorubicin is the only agent where increased surface density may account for it's increased potency in combination with an anti-EGF receptor antibody, but proposed to occur due to receptor block by antibody causing cell signal deprivation. The mechanism of cisplatin's increased potency is unclear and does not appear to be the result of effects on surface receptor density, but was proposed by Fan et al. to be cytoreduction and altered microenvironment (including tumor vascularity) interference with autocrine growth signals. In any event, these G₂/M-antibody combinations disclosed therein are disclaimed and do not form part of the present invention as defined by the appended claims.

The present invention is based, at least in part, on the observation that the therapeutic effectiveness of many therapeutic agents such as antibodies or small molecule therapeutics whose therapeutic effectiveness is, at least partly, based on the presence of an internalising cell surface structure, for example an antigen on the target cell may be enhanced through the use of a G₂/M agent.

Whilst not wishing to be bound by theory, it is believed that a cell treated with a G₂/M agent (and therefore blocked, or at least retarded, from further progression through the cell cycle) nevertheless continues to synthesise and present cell surface structures on its cell surface, leading to an increased density of the structure on the cell surface. It is believed that treatment of the cell with a G₂/M agent disrupts/perturbs/cripples (either temporarily or permanently) the internalisation mechanism of the cell. It is therefore believed that the increase in density of the cell surface structure is not as a result of increased gene expression per se but rather a combination of the continuance in protein synthesis and/or presentation by the cell and the effect on the internalisation mechanism.

Where these structures are attractive for therapeutic intervention, the subsequent therapeutic effectiveness of a therapeutic agent, such as an antibody or small molecule which targets that structure (e.g. by binding to it or otherwise interacting with the structure) is enhanced by virtue of an increased density in the target structure on the cell surface. This enhanced effect may take the form of improved efficacy of the therapeutic agent (e.g. increased tumour cell killing or induction of apoptosis in the cell expressing the target cell surface structure) or by attaining similar efficacy but at a lower effective dose of the therapeutic agent, potentially decreasing side effects for the patient. This enhanced effect is typically a result of synergistic or additive interaction between the G₂/M agent and the therapeutic agent.

The present inventors therefore teach that the blocking or retarding of a cell, particularly a cancerous cell, in the G₂/M phase of the cell cycle leads to an increase in density on the cell (i.e. plasma membrane) surface of a number of apparently unrelated antigens.

All citations and references appearing in this specification are expressly and entirely incorporated herein by reference.

### SUMMARY OF THE INVENTION

In accordance with the present invention there is provided a method of treating a mammalian patient, preferably human, in clinical need thereof which method comprises the step of simultaneously treating said patient with a G₂/M agent and a therapeutic agent whose therapeutic effectiveness depends at least in part on the expression on the cell surface of the patient cell of a cell surface structure that internalises as the cell progresses through its cell cycle (e.g. by binding to or otherwise interacting with the cell surface structure).

In accordance with the present invention there is provided a method of treating a mammalian patient, preferably human, in clinical need thereof which method comprises the step of simultaneously treating said patient with a G₂/M agent and a therapeutic agent wherein treatment with said G₂/M agent blocks or retards progression of the cell cycle in a target patient cell at G₂ and/or M thereby increasing the density of a cell surface structure in said target patient cell such as a protein or glycoprotein which structure is targeted by (e.g. specifically bound by) said therapeutic agent.

In accordance with the present invention, there is provided a method of treating a mammalian patient, preferably human, afflicted with a disease or disorder of cell cycle regulation (e.g. cancer) which method comprises the step of simultaneously treating said patient with a G₂/M agent and an agent whose therapeutic effectiveness depends at least partly, preferably mainly (even solely), on an internalising cell surface structure, particularly an internalising structure known or suspected to have a role in maintaining or progressing a cancerous state in said patient.

In accordance with another aspect of the present invention there is also provided a combination of a G₂/M agent and a therapeutic agent whose therapeutic effectiveness is based at least partly, preferably mainly, on the presence of an internalising cell surface structure.

Use of the combination as hereinbefore and hereinafter described in the manufacture of a medicament e.g. pharmaceutical preparation and in the treatment of a mammalian patient, particularly human is also provided.

The terms “block” and “arrest” are intended to be used interchangeably.

### DETAILED DESCRIPTION OF THE INVENTION

It will be apparent to those skilled in the art that the term “simultaneously treating” need not necessarily imply simultaneously administrating (although it does not exclude this). Indeed in many instances, it will be preferable to administer the G₂/M agent to the patient first, to block or retard cell cycle progression at G₂ and/or M to achieve the desired increase in cell surface structure density. This is then usually followed by exposing the same cells to the therapeutic agent that targets the cell surface structure thereby achieving enhanced therapeutic effectiveness of the therapeutic agent. The G₂/M agent may be administered on the same day as the therapeutic agent either together or within hours of each other. However, the G₂/M may also be administrated up to about two months beforehand, typically about one or two weeks beforehand and more typically less than a week beforehand, e.g. one to three days beforehand. Generally where the G₂/M agent has a known posology for monotherapeutic use, this maybe substantially followed prior to or together with administration of the therapeutic agent. Administration of the therapeutic agent may include multiple dosing (either as an oral medication, infusion or bolus dose) within several weeks after administration of the G₂/M agent (which itself may include multiple dosing either as an oral medication, infusion or bolus dose) but variation of this to take into account the respective pharmacokinetics and efficacy profile of the G₂/M agent and therapeutic agent may be required.

Treatment regimen is, of course, also dependent on a number of other factors such as the weight, age, general health status of the patient, type and severity of disease or disorder to be treated, all these being within the purview of the attending physician. Genetic predisposition of the patient to respond to treatment by a particular combination of the present invention may also require consideration. This may be achieved in advance of treatment by determining whether response is associated with a genetic polymorphism such as a gene region polymorphism e.g. single nucleotide polymorphism (SNP). The polymorphism is typically detected by directly determining the presence of the polymorphism sequence in a polynucleotide (e.g. genomic DNA or mRNA) or protein of the patient. Typically the presence of the polymorphism is determined in a method that comprises contacting a polynucleotide or protein of the patient with a specific binding agent for the polymorphism and determining whether the binding agent binds to a polymorphism in the polynucleotide or protein, the binding of the agent to the polymorphism indicating the likely response profile of the patient. The polymorphism maybe associated with metabolism (e.g. cytochrome P450 polymorphism) of any component of the combination.

The term “therapeutic effectiveness” or “therapeutically effective” or the like need not necessarily imply that the therapeutic agent is sufficiently effective to cure the disease or disorder. It is sufficient that therapeutic agent can ameliorate the disease or disorder state at least to some extent or otherwise provide a clinical benefit. Examples of treatment regimens that may be employed according to various aspects of the present invention are discussed in more detail below. However, it will be apparent that it is not an essential prerequisite that the mammalian patient is treated with at least two agents. The general principle is that target cells within the patient are arrested in preferably G₂ and/or M (or at least their progression through G₂ and/or M is retarded) which together with a therapeutic agent of the present invention has an enhanced therapeutic effectiveness. Thus a single agent that is able to fulfil both these roles are not necessarily excluded from the ambit of the present invention. It will also be apparent that precursor forms of the therapeutic agent and/or G₂/M agent are contemplated (that is forms of the agent which are therapeutically activated, by e.g. phase I metabolism, upon administration).

Treatment regimens involving one or more therapeutic agents and one or more G₂/M agents are envisaged. It will be apparent that the methods of the present invention may be used prophylatically where appropriate.

Cell Surface Structures

The term “cell surface structure” refers to structures that are present (e.g. expressed) on the cell surface of a cell and are anchored to the plasma membrane. Such structures may be proteins or modified proteins (such as glycoproteins) and are internalised by the cell, typically by the process of endocytosis, as the cell progresses through its cell cycle. Thus the term “internalising cell surface structure” refers to those cell surface structures that are internalised by the cell, typically by endocytosis, as the cell progresses through its cell cycle. The internalising cell surface structure is typically an antigen, transmembrane receptor (e.g. 7-transmembrane receptor) or other biological moiety which is synthesised and expressed by the cell and whose density at the cell surface may be increased by arresting the cell at G₂ and/or M (or at least retarding its progression therethrough). The term is not intended to extend to components of the plasma membrane itself, i.e. the lipid bilayer itself. The internalising cell surface structure may undergo various processing events prior to expression. A cell surface structure that is internalised may be determined through the use of an antibody specific for the suspected internalising cell surface structure (i.e. specifically binds thereto) which antibody is conjugated/coupled to a reporter moiety, i.e. a moiety whose presence can be detected according to conventional or available techniques. The reporter moiety may be, for example, a fluorescent dye or radioactive label. Detecting movement of the dye/marker into cells during cell culturing in the presence of the antibody/reporter moiety (which permits the antibody to bind to the suspected internalising cell surface structure) being indicative of an internalising cell surface structure. A suspected G₂/M agent of the present invention may be identified by observing increased binding of an antibody-/reporter moiety complex when the cell is arrested in G₂ and/or M. The particular stage that a cell is at during the cell cycle can be determined by known techniques well known to those skilled in the art, see for example Crissman et al, supra.

Thus, in accordance with the present invention there is provided a method for identifying a G₂/M agent which method comprises the steps of:

(a) providing a candidate agent;

(b) contacting said agent with preferably a mammalian cell, preferably a human cell, even more preferably a malignant mammalian cell;

(c) determining whether the density of a cell surface is increased;

(d) selecting said agent which causes said increase of step (c);

(e) optionally synthesising and/or purifying said agent of step (d).

In accordance with a further aspect, there is provided a method of treating a mammalian patient (afflicted with e.g. a disease of cell cycle regulation) in clinical need comprising the steps of;

(a) screening a candidate agent for the ability to increase the cell surface density of an internalising cell surface structure of a cell;

(b) selecting an agent which causes an increase in said cell surface density;

(c) simultaneously treating said patient with a therapeutically effective amount of; said agent of step (b) and a therapeutic agent which specifically binds to an internalising cell surface structure, preferably said structure of step.(a).

In accordance with the present invention there is provided a method for the treatment of a mammalian patient afflicted with a disease or disorder such as cancer, which method comprises the steps of;

(a) providing a G₂/M agent preferably by determining whether said agent has the ability to increase the cell surface density of an internalising cell surface structure;

(b) providing a therapeutic agent which specifically binds to or otherwise interacts with an internalising cell surface structure, preferably said structure of step (a) optionally by determining whether a candidate therapeutic agent binds to (e.g. specifically binds to) an internalising cell surface structure;

(c) simultaneously treating said patient with a therapeutically effective amount of; said G₂/M agent of step (a) and said therapeutic agent of step (b).

Internalising cell surface structures may comprise an extracellular, transmembrane and/or an intracellular portions. In many instances, the internalising cell surface structure (e.g. antigen) will comprise all three portions. Therapeutic agents of the present invention are those whose therapeutic effectiveness depends at least in part, preferably mainly, on the presence of an internalising structure on the cell surface. In many instances, this dependency will be as a result of the specific interaction (e.g. specific binding) of the therapeutic agent with the internalising cell surface structure. This binding may take place on the extracellular, transmembrane or intracellular portion of the cell surface structure. Preferably where the agent binds intracellularly, the agent binds to an intracellular catalytic domain of a protein (which will normally be coupled to the internal face of the plasma membrane or otherwise associated therewith). Examples of such proteins include those with kinase activity such as tyrosine kinase or serine/threonine kinase. Tyrosine kinase intracellular portions are a particularly attractive target for anti-cancer treatments. Of particular interest are tyrosine kinase inhibitors and in particular inhibitors of erB2 (or having a dual role in interacting with erB2 and EGFR see for example our co-pending PCT application WO 99/35146, the entire contents of which are incorporated herein by reference and to which the reader is specifically referred). Binding may then be followed by; the elicitation of a biological response to the binding e.g. ADCC, the inhibition of the catalytic properties of a protein, steric hindrance of the protein (for example by changing or interfering with the tertiary conformation of the protein) or competitive binding to an important effector site of the protein.

The internalising nature of the cell surface structure may therefore be utilised in the following general therapeutic approach. The increased cell surface structure density on the cell surface affords the opportunity of improving the delivery of a therapeutic agent into the target cell. The target cell is treated with a G₂/M agent to increase the density of the cell surface structure followed by treatment with the therapeutic agent. The treatment with the G₂/M agent is then stopped or reduced permitting the target cell, having the therapeutic agent bound to the internalising cell surface structure, to continue through its cell cycle and therefore internalise the therapeutic agent.

Internalising cell surface structures include those having a known or suspected disease association for example with a known or suspected role (e.g. causative role) in the initiation, maintenance or progression of a particular disease or disorder. Also included are those cell surface structures whose presence on the cell surface is indicative of a particular disease or disorder state. The present invention is of particular use in diseases of cell cycle regulation of which the best known are those having the collective term “cancer”. By increasing cell surface structure expression density at the cell surface, those structures that are normally presented at a relatively low density on the cell surface maybe presented at a higher, possibly more therapeutically useful density.

Internalising tumour cell surface structures (e.g. antigen) that may be targeted by the therapeutic agent (e.g. antibody) of the present invention include those having an established role in the initiation, progression or maintenance (or whose expression is indicative) of the cancerous state. These structures maybe mutated or otherwise altered forms of antigens expressed by normal cells, over expressed antigens or neoantigens, that is antigens expressed at an inappropriate point in the patients development. Examples of such antigens are c-erB2 (HER-2/neu), c-erbB3 (HER-3, Baulida J et al, J. Biological Chemistry 271(9), 5251-5257, 1996), c-erbB4 (HER-4, Baulida J et al, J. Biological Chemistry 271(9), 5251-5257, 1996), c-fins (Carlberg K et al, EMBO journal 10(4) 877-83, 1991) and the folate receptor (Lewis et al, Cancer Res, 58, 2952-2956).

Other examples of tumour antigens that may be targeted according to the present invention include: β integrin (J.Biological Chemistry 272(5): 2736-2743, 1997, Jan. 31), β2 integrins, e.g. Mac1/LFA1, Vascular Endothelial Growth Factor receptor 1 and 2 (VEGFR-1 and 2, Dougher, M., et al, Blood (1999) 81(10): 2767-2773), EDG-1 (Liu CH et al (1999) Mol.Biol.Cell, Apr.:10(4) 1179-90), Insulin growth factor (IGF-1) receptor (J.Biol.Chem. 1998 Nov. 27; 273(48):31640-3) and Prostate Specific Membrane Antigen (PSMA, Liu et al, Cancer Research 58, 4055-4060, 1998).

Other therapeutically useful target internalising antigens include those having known or suspected role in asthma and/or chronic obstructive pulmonary disorder (COPD). These therefore include: chemokine CCR3 (E1-Shazly A., et al Biochem.Biophys. Research Comm. (1999), 264:163-170), VLA, CXCR1 Barlic j. et al, J.Biol.Chem. 23(4):16287-16294), β2 integrin, P2Y₂ (Sromek, S. M. (1998) Molecular Pharmacology 54:485-494). The present invention also envisages improved treatments for diabetes mellitus by increasing cell surface density of the insulin receptor and in gene therapy where entry of the therapeutic genetic agent into the target cell is via a cell surface structure whose density can be increased by treatment with a G₂/M agent.

Therapeutic Agents

The therapeutic agent maybe an agonist, antagonist or mimetic of a particular cell function and may take the form of an antibody or other immunoglobulin (particularly when binding to the extracellular portion of the cell surface structure occurs), other protein or peptide species or otherwise a non-protein/non-peptide chemical entity (i.e. what is known in the art as a “small molecule”). The therapeutic agent delivered into the cell following internalisation according to the present invention is advantaegously cytotoxic leading to cell death (either apoptosis or necrosis). In the case where the therapeutic agent is an antibody specific for the internalising cell surface structure, a number of possible outcomes may occur following binding to the internalising cell surface structure depending, at least in part, on the effector function of the antibody. If the antibody has functional F_{c} function this may lead to the activation of complement-mediated cytotoxicity (CMC) and/or antibody dependent cell-mediated cytotoxicity (ADCC), either of which may result in lysis or phagocytosis of the target cell. In other embodiments, the antibody is conjugated to a therapeutically useful substance such as a radionuclide, enzyme or toxin as is well known and practised within the field.

The antibodies which specifically bind to an internalising cell surface structure e.g. antigen of the present invention preferably have the structure of a natural antibody or a fragment thereof. Antibodies typically comprise two heavy chains linked together by disulphide bonds and two light chains. Each light chain is linked to a respective heavy chain by disulphide bonds. Each heavy chain has at one end a variable domain followed by a number of constant domains. Each light chain has a variable domain at one end and a constant domain at its other end. The light chain variable domain is aligned with the variable domain of the heavy chain. The light chain constant domain is aligned with the first constant domain of the heavy chain. The constant domains in the light and heavy chains are not involved directly in binding the antibody to antigen.

The variable domains of each pair of light and heavy chains form the antigen binding site. The domains on the light and heavy chains have the same general structure and each domain comprises a framework of four regions, whose sequences are relatively conserved, connected by three complementarity determining regions (CDRs). The four framework regions largely adopt a beta-sheet conformation and the CDRs form loops connecting, and in some cases forming part of, the beta-sheet structure. The CDRs are held in close proximity by the framework regions and with the CDRs from the other domain, contribute to the formation of the antigen binding site, which in the case of the present invention is the formation of an internalising antigen binding site. CDRs and framework regions of antibodies may be determined by reference to Kabat et al (“Sequences of proteins of immunological interest” U.S. Dept. of Health and Human Services, U.S. Government Printing Office, 1987).

The preparation of an antibody in which the CDRs are derived from a different species than the framework of the antibody's variable domains is disclosed in EP-A-0239400. The CDR's may be derived from a rodent or primate monoclonal antibody. The framework of the variable domains and the constant domains of such altered antibodies are usually derived from a human antibody. Such a humanised antibody should not elicit as great an immune response when administered to a human compared to the immune response mounted by a human against a wholly foreign antibody such as one derived from a rodent.

The antibody preferably has the structure of a natural antibody or a fragment thereof. Throughout the specification reference to antibody therefore comprises not only a complete antibody but also fragments such as a (Fab′)₂ fragment, a Fab fragment, a light chain dimer or a heavy chain dimer. The antibody may be an IgG such as IgG₁, IgG₂, IgG₃ or IgG₄; or IgM, IgA, IgE or IgD or a modified variant thereof, including those that may be conjugated to other molecules such as radionuclides, enzymes etc. Typically, the constant region is selected according to the functionality required. Normally an IgG1 will demonstrate lytic ability through binding to complement and will mediate ADCC (antibody dependent cell cytotoxicity). An IgG₄ antibody will be preferred if a non-cytotoxic antibody is required. Antibodies according to the present invention also include bispecific antibodies. Antibodies of the present invention may be murine, chimaeric or humanised with the preferred antibody being humanised antibody.

There are four general steps to humanise a monoclonal antibody. These are:

(1) determining the nucleotide and predicted amino acid sequence of the starting antibody light and heavy variable domains;

(2) designing the humanised antibody, i.e. deciding which antibody framework region to use during the humanising process;

(3) the actual humanising methodologies/techniques; and

(4) the transfection and expression of the humanised antibody.

More specifically,

Step 1: Determining the Nucleotide and Predicted Amino Acid Sequence of the Antibody Light and Heavy Chain Variable Domains

To humanise an antibody only the amino acid sequence of the antibody's heavy and light chain variable domains needs to be known. The sequence of the constant domains is irrelevant because these do not contribute to the reshaping strategy. The simplest method of determining an antibody variable domain amino acid sequence is from cloned cDNA encoding the heavy and light variable domain.

There are two general methods for cloning a given antibody's heavy and light chain variable domain cDNAs: (1) via a conventional cDNA library, or (2) via the polymerase chain reaction (PCR). Both of these methods are widely known. Given the nucleotide sequence of the cDNAs, it is a simple matter to translate this information into the predicted amino acid sequence of the antibody variable domains.

Step 2: Designing the Humanised Antibody

There are several factors to consider in deciding which human antibody sequence to use during the humanisation. The humanisation of light and heavy chains are considered independently of one another, but the reasoning is basically similar for each.

This selection process is based on the following rationale: a given antibody's antigen specificity and affinity is primarily determined by the amino acid sequence of the variable region CDRs. Variable domain framework residues have little or no direct contribution. The primary function of the framework regions is to hold the CDRs in their proper spatial orientation to recognise the antigen. Thus the substitution of rodent CDRs into a human variable domain framework is most likely to result in retention of their correct spatial orientation if the human variable domain framework is highly homologous to the rodent variable domain from which they originated. A human variable domain should preferably be chosen therefore that is highly homologous to the rodent variable domain(s).

A suitable human antibody variable domain sequence can be selected as follows:

1. Using a computer program, search all available protein (and DNA) databases for those human antibody variable domain sequences that are most homologous to the rodent antibody variable domains. The output of a suitable program is a list of sequences most homologous to the rodent antibody, the percent homology to each sequence, and an alignment of each sequence to the rodent sequence. This is done independently for both the heavy and light chain variable domain sequences. The above analyses are more easily accomplished if only human immunoglobulin sequences are included.

2. List the human antibody variable domain sequences and compare for homology. Primarily the comparison is performed on lengths of CDRs, except CDR 3 of the heavy chain which is quite variable. Human heavy chains and Kappa and Lambda light chains are divided into subgroups; Heavy chain 3 subgroups, Kappa chain 4 subgroups, Lambda chain 6 subgroups. The CDR sizes within each subgroup are similar but vary between subgroups. It is usually possible to match a rodent antibody CDR to one of the human subgroups as a first approximation of homology. Antibodies bearing CDRs of similar length are then compared for amino acid sequence homology, especially within the CDRs, but also in the surrounding framework regions. The human variable domain which is most homologous is chosen as the framework for humanisation.

Step 3: The Actual Humanising Methodologies/Techniques

An antibody may be humanised by grafting the desired CDRs onto a human framework according to EP-A-0239400.(see also P. T. Jones et al, Nature 321:522 (1986); L. Reichman et al, Nature 332 :323(1988); Verhoeyen M. et al, Science 239:1534 (1988) and J. Ellis et al, The Journal of Immunology, 155 :925-937(1995)). A DNA sequence encoding the desired reshaped antibody can therefore be made beginning with the human DNA whose CDRs it is wished to reshape. The rodent variable domain amino acid sequence containing the desired CDRs is compared to that of the chosen human antibody variable domain sequence. The residues in the human variable domain are marked that need to be changed to the corresponding residue in the rodent to make the human variable region incorporate the rodent CDRs. There may also be residues that need substituting in, adding to or deleting from the human sequence.

Oligonucleotides are synthesised that can be used to mutagenise the human variable domain framework to contain the desired residues. Those oligonucleotides can be of any convenient size. One is normally only limited in length by the capabilities of the particular synthesiser one has available. The method of oligonucleotide-directed in vitro mutagenesis is well known.

Alternatively humanisation may be achieved using the recombinant polymerase chain reaction (PCR) methodology of WO92/07075. Using this methodology, a CDR may be spliced between the framework regions of a human antibody.

In general, the technique of WO92/07075 can be performed using a template comprising two human framework regions, AB and CD and between them, the CDR which is to be replaced by a donor CDR. Primers A and B are used to amplify the framework region AB, and primers C and D used to amplify the framework region CD. However, the primers B and C each also contain, at their 5′ ends, an additional sequence corresponding to all or at least part of the donor CDR sequence. Primers B and C overlap by a length sufficient to permit annealing of their 5′ ends to each other under conditions which allow a PCR to be performed. Thus, the amplified regions AB and CD may undergo gene splicing by overlap extension to produce the humanised product in a single reaction.

Step 4: The Transfection and Expression of the Reshaped Antibody

Following the mutagenesis reactions to reshape the antibody, the mutagenised DNAs can be linked to an appropriate DNA encoding a light or heavy chain constant region, cloned into an expression vector, and transfected into host cells, preferably mammalian cells. These steps can be carried out in routine fashion. A reshaped antibody may therefore be prepared by a process comprising:

(a) preparing a first replicable expression vector including a suitable promoter operably linked to a DNA sequence which encodes at least a variable domain of an Ig heavy or light chain, the variable domain comprising framework regions from a human antibody and the CDRs required for the humanised antibody of the invention.

(b) preparing a second replicable expression vector including a suitable promoter operably linked to a DNA sequence which encodes at least the variable domain of a complementary Ig light or heavy chain respectively;

(c) transforming a cell line with the first or both prepared vectors; and

d) culturing said transformed cell line to produce said altered antibody.

Preferably the DNA sequence in step (a) encodes both the variable domain and the or each constant domain of the human antibody chain. The humanised antibody can be recovered and purified. The cell line which is transformed to produce the altered antibody may be Chinese Hamster Ovary (CHO) cell line or an immortalized mammalian cell line, which is advantageously of lymphoid origin, such as a myeloma, hybridoma, trioma or quadroma cell line. The cell line may also comprise a normal lymphoid cell, such as a B-cell, which has been immortalised by transformation with a virus, such as the Epstein-Barr virus. Most preferably, the immortalised cell line is a myeloma cell line or a derivative thereof. The expression system of choice is the glutamine synthetase expression system described in WO87/00462 (see also P. E. Stephens et al, Nucleic Acid Res. 17:7110 (1989) and C. R. Bebbington et al, Bio/Technology 10:169 (1992)).

Although the cell line used to produce the humanised antibody is preferably a mammalian cell line, any other suitable cell line, such as a bacterial cell line or a yeast cell line, may alternatively be used. For single antibody chains, it is envisaged that E. coli—derived bacterial strains could be used. The antibody obtained is checked for functionality. If functionality is lost, it is necessary to return to step (2) and alter the framework of the antibody.

Once expressed, the whole antibodies, their dimers, individual light and heavy chains, or other immunoglobulin forms of the present invention can be purified according to standard procedures of the art, including ammonium sulfate precipitation, affinity columns, column chromatography, gel electrophoresis and the like (see generally Scopes, R, Protein Purification, Springer-Verlag, N.Y. (1982)). Substantially pure immunoglobulins of at least about 90 to 95% homogeneity are preferred and 98 to 99% or more homogeneity most preferred, for pharmaceutical uses. Once purified, partially or to homogeneity as desired, an antibody may then be used therapeutically.

G₂/M Agents

G₂/M agents of the present invention are capable of affecting cell growth by blocking (or retarding) progression of the cell cycle G₂ and/or M. Examples of G₂/M agents which are capable of blocking (or retarding) cell cycle progression in G₂ and/or M are vinorelbine, cisplatin, mytomycin, paclitaxel, carboplatin, oxaliplatin and CPT-II (camptothecin).

The dose and regimen employed according to the present invention may be the same or substantially similar to an established dose and regimen for that G₂/M agent. Optimisation however for reasons such as severity and type of the disease or disorder to be treated is taught.

Vinorelbine tartrate is a semisynthetic vinca alkaloid with the chemical name 3′, 4′-didehydro-4′-deoxy-C′-norvincaleukoblastine [R-(R*,R*)-2,3-dihydroxybutanedioate (1:2)(salt)]. Vinorelbine tartrate is used in combination with other chemotherapy agents such as cisplatin or as a single agent in the treatment of various solid tumours particularly non-small cell lung, advanced breast, and hormone refractory prostate cancers. The brand name Navelbine® is used in North America and Europe. Navelbine® is administered intravenously as a single-agent or in combination therapy typically at doses of 20-30 mg/m² on a weekly basis. An oral formulation of vinorelbine is in clinical development.

Cisplatin has the chemical name cis-diamminedichloroplatinum. Cisplatin is used in the treatment of metastatic testicular tumours as a combination therapy, as single and combination therapy in metastatic ovarian tumours, as well as a single agent in advanced bladder cancer. Cisplatin is manufactured by Bristol-Myers Squibb under the brand names of Platinol® and Platinol-AQ®. Cisplatin is also used in the following types of cancer, typically in combination therapy: non-small cell and small cell lung cancers, head and neck, endometrial, cervical, and non-Hodgkin's lymphoma. Cisplatin is typically administered intravenously in doses ranging from 15-150 mg/m² once every 3 to 4 weeks, or daily for 5 days repeated every 3 or 4 weeks. However, higher and more frequent doses are occasionally administered and the route of administration could be different than intravenous, such as intra-arterial or intraperitoneal.

Carboplatin has the chemical name platinum, diammine [1,1-cyclobutane-dicarboxylato(2)-0,0′]-(SP-4-2). Carboplatin is usually administered in combination with other cytotoxics such as paclitaxel and etoposide. It is used in the treatment of advanced ovarian cancer, non-small cell lung cancer as well as in many of the same types of cancer as cisplatin is used. The brand name of carboplatin manufactured by Bristol-Myers Squibb is Paraplatin®. Carboplatin is typically administered intravenously at 300-400 mg/m², or to a target area under the drug concentration versus time curve (AUC) of 4-6 mg/ml-min using the patient's estimated glomerular filtration rate (GFR). Higher doses up to around 1600 mg/m² divided over several, usually five, days may also be administered.

Paclitaxel has the chemical name 5β, 20 epoxy-1,2α,4,7β,10β,13α-hexahydroxytax-11-en-9-one 4,10-diacetate 2-benzoate 13-ester with (2R, 3S)-N-benzoyl-3-phenylisoserine. Paclitaxel is manufactured by Bristol-Myers Squibb as Taxol®. It is used to treat a variety of carcinomas including ovarian, breast, non-small cell lung, head and neck. Typical doses include 135-175 mg/m² as either a 3 or 24 hour intravenous infusion given every 3 or 4 weeks. Higher doses up to around 300 mg/m2 have also been administered.

Besides the active ingredient, the drug products provided by manufacturers typically contain a diluent such as sterile water, dextrose 5% in water or 0.9% sodium chloride in water with additional excipients such as Cremophor vehicle added to make for example, paclitaxel soluble.

Other G₂/M agents that may block or retard progression of the cell cycle in G₂ and/or M include anthracyclines e.g. doxorubicin and aclarubicin; carmustine (BCNU), camptothecin, 9-nitro-camptothecin, cyclophosphamide and its derivatives, docetaxel, etoposide, Razoxane (ICRF-187), alkyllyso-phospholipids e.g. ilmofosine; methotrexate, MST-16, taxanes, vinblastine, vincristine and teniposide (VM-26) (again see Martindale, The Extra Pharmacopoeia, 31st edition, edited by JEF Reynolds, London, Royal Pharmaceutical Society, 1996,) and flavonoids e.g. apigenin and genistein (see The Merck Index, 12th edition, Merck Research Laboratories, Merck and Co Inc, 1996). In addition, adozelesin (a class of pyrazole compounds) (Cancer Research 1992, Oct. 15; 52 (2): 5687 to 5692)), Bistratene A (Mutation Research 1996, Mar. 1; 367 (3): 169 to 175), cycloxazoline (Cancer Chemotherapy & Pharmacology 1994; 33(5): 399 to 409), imidazoarcridinone, melephan (Experimental Cell Biology 1986; 54 (3): 138 to 148 and International Journal of Cancer 1995, Jul. 17; 62 (2): 170 to 175), merbarone (Environmental & Molecular Mutagenesis 1997; 29 (1): 16 to 27 and Cancer Research 1995, Apr. 1; 55 (7): 1509 to 1516) and oracin (FEBS Letters 1997, Jan. 2; 400 (1): 127 to 130) are also believed to block (or retard) cell cycle progression in G₂ and/or M. Generally all topo II inhibitors, e.g. to potecan (abpi, 1998-1999), all vinca derivatives and all DNA damaging agents including radiation are also believed to arrest cells in G₂ and/or M. Further examples include RAF kinase inhibitors (see for example, Clinical Can.Res 4(5):1111-1116, May 1998 and our co-pending application WO 99/10325, the entire contents of which are incorporated herein by reference and to which the reader is specifically referred).

Moreover, 5FU has been reported to arrest cells in G₂ and/or M (Oncology Research 1994; 6(7):303-309) and it is therefore believed that 5FU and compounds similar to 5FU such as UFT, methotrexate, capecitabine and Gemcitabine will increase internalising antigen expression in some tissues. Similarly, tomudex (Raloxifen) which is known to arrest cells in the S phase is believed to increase internalising antigen expression.

The term “G₂/M agent” is therefore not limited to cytotoxic therapy, but also encompasses cytostatic therapy and any other drugs capable of blocking (or retarding) cell cycle progression in G₂ and/or M. Combinations of drugs which together result in blocking or retarding cell progression at or through G₂/M are contemplated. Throughout the specification reference to a G₂/M agent includes combinations of one or more specific chemotherapeutic agents which arrest (or retard) internalising cell surface structure expressing cells (particularly tumour antigens) in G₂ and/or M. Examples of typical combinations are vinorelbine with cisplatin and paclitaxel with carboplatin; oxaliplatin with 5FU; cyclophosphamide with methotrexate and 5FU; cyclophosphamide with doxorubicin and 5FU.

While it is possible for the G₂/M agent to be administered alone it is preferable to present it as a pharmaceutical composition comprising an active ingredient, as defined above, together with an acceptable carrier therefor. Each carrier must be “acceptable” in the sense of being compatible with the other ingredients of the composition and not injurious to the recipient.

Therapeutic Protocols (or Regimens)

Preferred dosing schedules for administration of the G₂/M agent and therapeutic agent (particularly where the therapeutic agent is an antibody or other immunoglobulin) include: administering the therapeutic agent once every one or two weeks, preferably once every three or four weeks or a combination thereof for as long as necessary. The G₂/M agent is given according to the established regimen for that agent or a regimen which will allow exposure of internalising cell surface structure expressing cells to be blocked/arrested or retarded in G₂/M. Preferred dosing schedules vary with the therapeutic agent and disease state but include, for example, once weekly, once every three or four weeks, or daily for several (e.g. 3-5) days repeated every three or four weeks for as long as necessary. Dosing of the therapeutic agent may take place on the same day or different days as indicated for the G₂/M agent. Adjustment of the dosing schedule or strength of dose to prevent or decrease toxicity or side effects may take place with either the therapeutic agent or the G₂/M agent.

For example, the preferred dosing schedule for co-administration of vinorelbine and cisplatin in combination with a therapeutic agent which binds the target internalising cell surface structure (e.g. an antibody) is administration of the agent at a dose supported by clinical studies e.g. 30 mg/m² once a week for as long as necessary but typically for a period of 3 to 4 weeks, followed by a 30 mg/m² dose every other week thereafter for as long as necessary. Vinorelbine is administered at a dose 25 mg/m² on day 1,8,15 and 22. Cisplatin is given only once at a dose of 100 mg/m² on day 1. Thereafter the vinorelbine/cisplatin regime is repeated every 28 days for as long as necessary. Preferably, vinorelbine, cisplatin and the antibody are administered at the same time on day one over a period of about 2 to 3 hours.

Another example of a preferred dosing schedule is the administration of paclitaxel/carboplatin in combination with the therapeutic agent (e.g. antibody) is administered as for the vinorelbine/cisplatin example above and paclitaxel and carboplatin are given at a dose of 225 mg/m² and AUC=6.0 respectively, on day 1, with a repeat dosage every 28 days thereafter for as long as necessary. Again, paclitaxel, carboplatin and the antibody are preferably administered together on day 1 over a period of about 2 to 3 hours.

Other preferred dosage schedules which comprise the combination of the antibody with any of navelbine, cisplatin or taxol on their own would comprise similar dosages and administration schedules, using just one anticancer agent instead of two.

Preferred combinations of a therapeutic agent and a G₂/M agent are: The therapeutic agent in combination with any of the following chemotherapeutic agents: UFT, Capecitabine, CPT-II, Oxaliplatin, 5FU, 5FU continuous infusion, Paclitaxel, Docetaxel, Cyclophosphamide, Methotrexate, Doxorubicin, Navelbine (iv and oral), Epirubicin, Mitoxantrone, Raloxifen, Cisplatin, Mitomycin, Carboplatinum, Gemcitabine, Etoposide and Topotecan.

Particularly preferred combinations are the therapeutic agent with CPT-II, 5FU (continuous infusion), Oxaliplatin, Capecitibine, UFT and Tomudex (Raloxifen).

These combinations are useful in the treatment of cancer, particularly in the treatment of colorectal cancer, breast cancer, gastric cancer, prostate cancer and non-small-cell lung cancer.

Specifically, the following combinations are particularly preferred for colorectal cancer: the therapeutic agent in combination with: UFT (optionally with Leucovorin); Capecitabine; Oxaliplatin (optionally with 5FU); CPT-II, 5FU (optionally with Eniluracil or Levamisole or Leucovorin); 5FU protracted continuous infusion; and Mitomycin.

Preferred combinations for the treatment of breast cancer are: the therapeutic agent in combination with Paclitaxel; Docetaxel; Cyclophosphamide (optionally with 5FU and either Methotrexate or Doxorubicin); Navelbine (iv and/or oral); Doxorubicine; Epirubicin; Mitoxantrone; and tomudex.

Preferred combinations for the treatment of gastric cancer are: the therapeutic agent in combination with Cisplatin; 5FU; Mitomycin; and Carboplatinum.

A preferred combination for the treatment of prostatic cancer is: the therapeutic agent in combination with Mitoxantrone.

Preferred combinations for the treatment of non-small-cell lung cancer are: the therapeutic agent in combination with: Navelbine; Cisplatin; Carboplatin; Paclitaxel; Docetaxel; Gemcitabine; Topotecan; and Etoposide.

More detailed information on treatment regimens, dosages and compositions etc can be obtained from standard reference books such as: Martindale, The Extra Pharmacopoeia, 31st edition, edited by J E F Reynolds, London, Royal Pharmaceutical Society, 1996 and the Physicians Desk reference, 49th Edition, 1995, Medical Economics Data Production Company, Montvale.

Pharmaceutical Preparations

Pharmaceutical preparations of the present invention include those suitable for oral, rectal, nasal, topical (including buccal and sublingual), vaginal, parenteral (including subcutaneous, intramuscular, intravenous and intradermal) or transdermal administration. The preparations may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product. The preparation may comprise the G₂/M agent and therapeutic agent as separate compositions suitable of administration or combined into a single composition ready for administration.

Preparations of the G₂/M agent suitable for oral administration may be presented as discrete units such as capsules, cachets or tablets each containing a predetermined amount of the active ingredient; as a powder or granules; as a solution or suspension in an aqueous or non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropylmethyl cellulose), lubricants, inert diluent, preservative, disintegrant (eg. sodium starch glycollate, cross-linked povidone, cross-linked sodium carboxymethyl cellullose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating to provide release in parts of the gut other than the stomach.

Preparations suitable for oral use as described above may also include buffering agents designed to neutralise stomach acidity. Such buffers may be chosen from a variety of organic or inorganic agents such as weak acids or bases admixed with their conjugated salts.

Preparations suitable for topical administration in the mouth include lozenges comprising the active ingredient in a flavoured basis, usually sucrose and acacia or tragacanth; pastilles comprising the active ingredient in an inert basis such as gelatine and glycerin, or sucrose and acacia and mouthwashes comprising the active ingredient in a suitable carrier.

Preparations for rectal administration may be presented as a suppository with suitable base comprising for example cocoa butter or a salicylate.

Preparations suitable for vaginal administration may be presented as pessaries, tampons, creams, gels, pastes, foams or spray formulations containing in addition to the active ingredient such carriers as are known in the art to be appropriate.

Preparations suitable for parenteral administration include aqueous and non-aqueous isotonic sterile injection solutions which may contain anti-oxidants, buffers, bacteriostats and solutes which render the compositions isotonic with the blood of the intended recipient; and aqueous and non-aqueous sterile suspensions which may include suspending agents and thickening agents, such as liposomes or other microparticulate systems which are designed to target the compounds to blood components or one or more organs. The preparations may be presented in unit-dose or multi-dose sealed containers, for example, ampoules and vials, and may be stored in a freeze-dried (lyophilized) condition requiring only the addition of sterile liquid carrier, for example water for injections, immediately prior to use. Extemporaneous injection solutions and suspensions may be prepared from sterile powders, granules and tablets of the kind previously described.

Preparations suitable for transdermal administration may be presented as discrete patches adapted to remain in intimate contact with the epidermis of the recipient for a prolonged period of time. Such patches suitably contain the active ingredient as an optionally buffered, aqueous solution of, for example, 0.1-0.2M concentration with respect to said compound. As one particular possibility, the active ingredient may be delivered from the patch by iontophoresis as generally described in Pharmaceutical Research, 3 (6),318 (1986).

It should be understood that in addition to the ingredients particularly mentioned above the compositions in question, for example, those suitable for oral administration may include such further agents as sweeteners, thickeners and flavouring agents.

In accordance with another aspect of the present invention there is provided a pharmaceutical preparation comprising a G₂/M agent and an therapeutic agent preferably together with instructions for administrating the preparation to a mammalian patient, preferably human (i.e. instructions for carrying out a medical treatment, particularly a treatment for a disease of cell cycle regulation such as cancer or particular diseases or disorders that the preparation is useful for/approved for). Simultaneous treatment of the patient in accordance with the instructions may lead to a biological interaction within the patient between the G₂/M agent and the therapeutic agent which interaction has enhanced therapeutic effect (e.g. additive or synergistic effect). In many instances the interaction between the G₂/M agent and therapeutic agent can be determined as enhanced (e.g. additive, but preferably synergistic) by comparing the effectiveness of the simultaneous treatment of G₂/M agent and the therapeutic agent on the one hand and the effectiveness of non-simultaneous treatment. The definition of “additive” and “synergistic” being terms of the art.

In accordance with another aspect of the present invention, there is provided a method of treating a mammalian patient afflicted with cancer which method comprises the step of simultaneously treating said patient with cytotoxic agent and a cytostatic agent, particularly one which blocks or retards cell cycle progression in G₂ and/or M.

In accordance with another aspect of the present invention there is provided a method of treating a mammalian patient, particularly human patient afflicted with a disease of cell cycle regulation e.g. cancer which method comprises the step of simultaneously treating said patient with a G₂/M agent and a therapeutic agent (e.g. antibody or small molecule) which is capable of specifically binding an internalising antigen presented on the cell surface of the diseased (i.e cancerous) cell.

In accordance with another aspect of the present invention there is provided a method of treating a mammalian patient afflicted with a disease of cell cycle regulation, e.g. cancer which method comprises the step of simultaneously treating said patient e.g. human with a G₂/M agent and a therapeutic agent, preferably a non-protein/non-peptide chemical agent, which therapeutic agent targets (e.g. binds to and/or inhibits the function of) one or more of the following:

A tyrosine kinase e.g. erbB2 or VEGFr-2.

In accordance with another aspect of the present invention there is provided a method of treating a mammalian patient e.g. human afflicted with a disease of cell cycle regulation such as cancer, which method comprises the step of simultaneously treating said patient with an effective amount of a G₂/M agent and an effective amount of a therapeutic agent which binds to an internalising antigen (either at the extracellular, transmembrane or intracellular portion of the internalising antigen) thereby bringing about a therapeutic effect on said patient.

In accordance with another aspect of the present invention there is provided a method of treating a mammalian patient e.g. human afflicted with a disease of cell cycle regulation such as cancer, which method comprises the step of simultaneously treating said patient with an effective amount of a G₂/M agent and an effective amount of a therapeutic agent which therapeutic agent binds to an internalising antigen (either at the extracellular, transmembrane or intracellular portion of the internalising antigen), said G₂/M agent and therapeutic agent interacting within said patient, said interaction having a synergistic therapeutic effect on said patient.

In accordance with the present invention there is provided a combination of a G₂/M agent and a therapeutic agent whose therapeutic effectiveness depends on the expression of an internalising cell surface structure such as an antigen on a target diseased cell which internalisation is capable of being blocked or impeded by said G₂/M agent.

In accordance with the present invention there is provided a combination of a G₂/M agent and a therapeutic agent whose therapeutic effectiveness depends at least in part on the expression of an internalising cell surface structure such as an antigen whose density at the cell surface can be increased by treatment of the cell with the G₂/M agent.

In accordance with the present invention there is provided a kit-of-parts comprising a G₂/M agent and a therapeutic agent whose therapeutic effectiveness depends, at least in part, on the presence of an internalising cell surface structure on a target cell.

The present invention is defined by the appended claims with the proviso that the G₂/M agent and therapeutic agent in combination (whether described as a combination or not) is not:

Ep-CAM specific antibodies together with a G₂/M agent.

Monoclonal antibodies such as disclosed in WO 89/06692 (specifically Herceptin®, otherwise known as trastuzumab or rhuMab) together with Taxol, docetaxel or

Navelbine as the G₂/M agent.

Navelbine together with Taxol as the G₂/M agent.

Agents that target Epidermal growth factor receptor (EGFr) and a G₂/M agent.

The present invention will now be described by way of example only. These are for exemplary purposes only and are not intended to limit the invention in any way.

In the Figures:

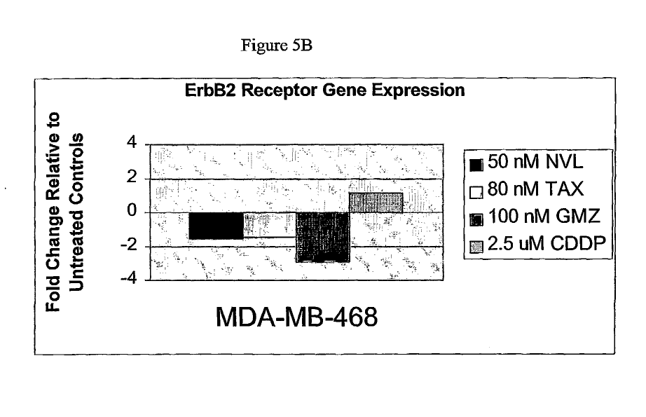

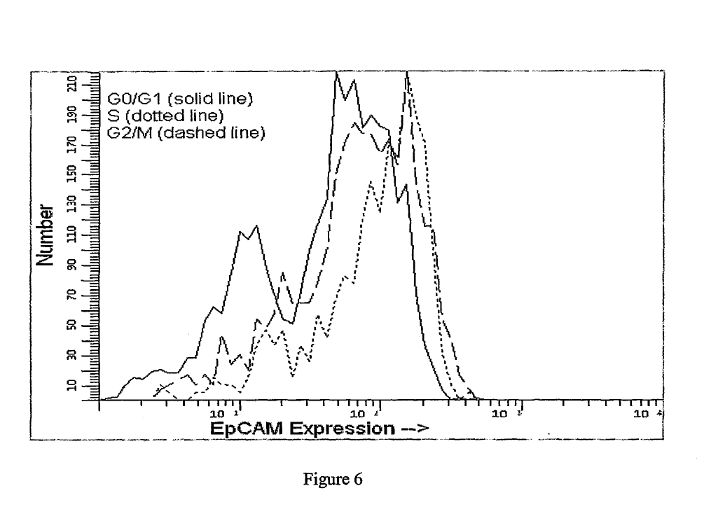

FIG. 6. Populations of PC-3 prostatic adenocarcinoma cells in culture were evaluated for distribution in G₀/G₁ (solid line), S (dotted line), and G₂/M (dashed line) phases of cell cycle and characterized for Ep-CAM antigen expression at each phase. Ep-CAM is expressed at higher density and homogeneity in S and G₂/M phases.

Antigen expression varied by phase across the cell cycle on PC-3 prostastic adenocarcinoma cells. Populations of PC-3 prostatic adenocarcinoma cells were evaluated for distribution in G₀/G₁, S and G₂/M phases of the cell cycle as well as Ep-CAM expression of the cell surface. FIG. 6 demonstrates that Ep-CAM is expressed across the cell cycle, but at higher density and greater homogeneity in cells in S and in G₂/M phases than G₀/G₁. This pattern of expression has been documented in a number of other human colon, prostate, and lung tumor cells in culture.

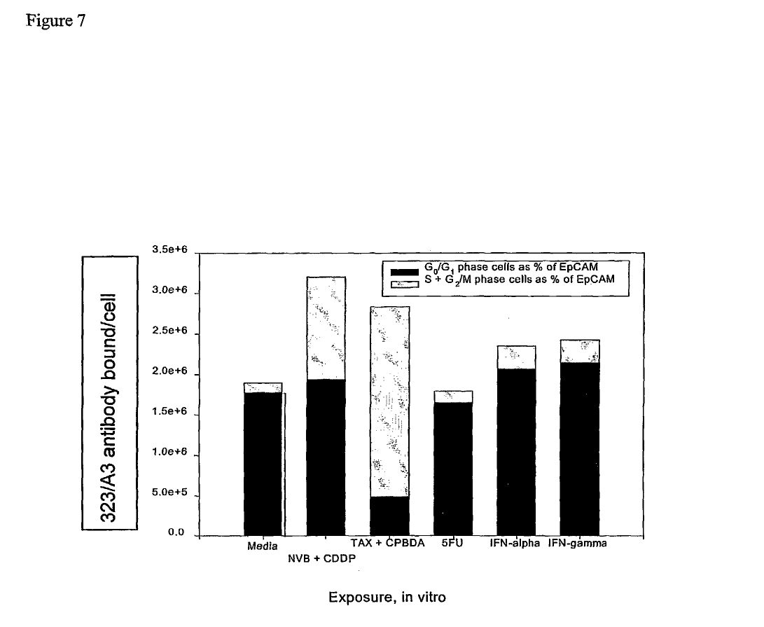

FIG. 7. Cell Cycle Analysis and Quantitation of Antigen Expression. Populations of adenocarcinoma cells were evaluated for distribution in G₀/G₁, S, and G₂/M phases of the cell cycle as well as Ep-CAM presentation on the cell surface. Subconfluent cells were exposed to Navelbine or Taxol for up to 24 hours, then washed and exposed to cisplatin or carboplatin, respectively, overnight. Cells were exposed to 5-fluorouracil (5-FU) for 24 hours, and to the interferons continuously for 2-5 days. Cells were washed and cultured for another 2 days prior to analysis for antigen presentation except for cells exposed to interferons. Cells were lightly trypsinized and mechanically detached from the culture flasks and resuspended in calcium- and magnesium-free phosphate-buffered saline containing bovine serum albumin and sodium azide. Exactly 2×10⁵ cells were stained with FITC-323/A3 murine IgG antibody or FITC-murine IgG (control). Cells were fixed with cold paraformaldehyde, then permeabilized for DNA staining with Tween-20. Cellular DNA was stained with a propidium iodide buffer containing RNAse A. Listmode data were acquired using Lysis II software on a FACScan flow cytometer (Becton Dickinson Immunocytometry Systems) equipped with a 488 nm laser. Cell cycle analysis was done using CellFit software for SOBR modeling of the histograms (where possible, otherwise manual estimations were employed) on Cell-Fit. Ep-CAM antigen presentation was quantitated by comparison of the mean fluorescence intensity of fluorescein-conjugated 323/A3 bound to cultured cells with the fluorescence intensity of calibrated microbead standards and evaluated separately using histogram analysis in WinList (Verity Software House). Standard curves of calibration bead concentration versus fluorescence intensity were constructed in SoftMax Pro (Molecular Devices, Inc.), and fluorescence intensity of stained cells was used to calculate the number of antigen molecules per cell for the population.

Increased expression of Ep-CAM antigen on HT-29 colon adenocarcinoma cells in culture following pretreatment with chemotherapeutic agents was associated with arrest of cell cycle progression and accumulation of cells in S and G₂/M phases. Adenocarcinoma cells (HT-29) were exposed to Navelbine or Taxol or combinations of drugs as indicated in FIG. 7 and the cells were evaluated for cell surface Ep-CAM presentation in addition to cell cycle distribution. Cell cycle analysis demonstrated that only 6.3% of the media control cells were in S and G₂/M phases combined, compared to 39.4% of the Navelbine followed by cisplatin (CDDP) combination and 82.6% of Taxol followed by carboplatin (CPBDA) combination. More importantly, both drug combinations caused significant increases in cell surface Ep-CAM expression. Antigen expression was not significantly increased in cells exposed to 5-FU, interferon-alpha, or interferon-gamma, which had only 7.9%, 12%, and 11.5%, respectively, of cells in S and G₂/M phase. Thus, only the drugs that caused accumulation of cells in S or G₂/M phases were able to produce a significant increase in Ep-CAM antigen presentation. It has reported in the literature that interferons cause an increase in cell surface presentation of certain antigens by exerting their affect at the level of gene expression. Our results are consistent with the published results of others (Shimada, S., Ogawa, M., Schlom, J. and Greiner J. W. Comparison of the Interferon-γ-Mediated Regulation of Tumor-Associated Antigens Expressed by Human Gastric Carcinoma Cells. in vivo 7:1-8, 1993.) and have shown that interferons have no influence on cell surface presentation of Ep-CAM.

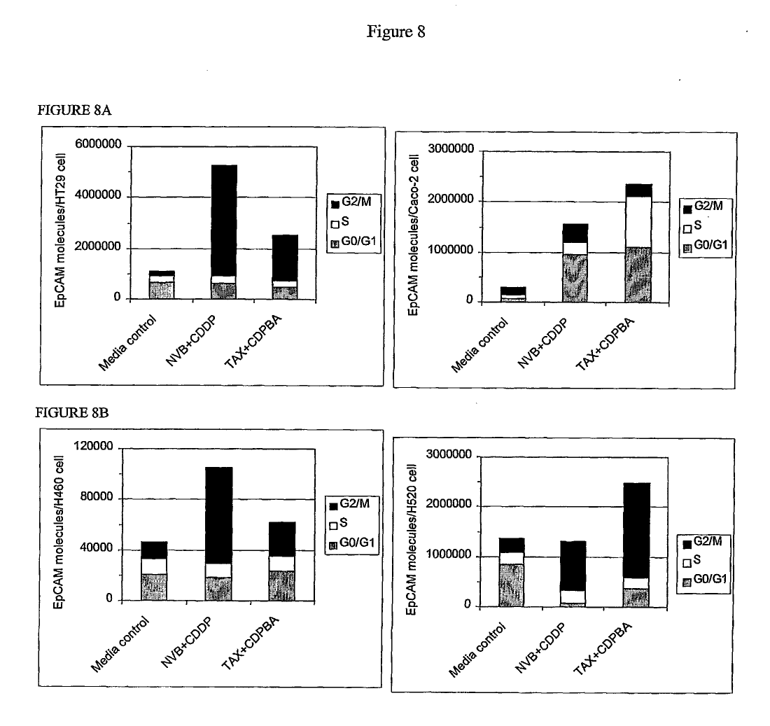

FIG. 8. The cell surface quantitation of Ep-CAM antigen and cell cycle distribution from various human colon (A, B), lung (C, D) adenocarcinoma cells in culture. Cells were exposed to Navelbine (NVL; 30 nM) plus cisplatin (CDDP; 5 μM), or Taxol (TAX; 80 nM) plus carboplatin (CBPDA; 100 μM) and compared to media alone. The area of each bar is divided to indicate the percentage of cells in G₀/G₁, S, and G₂/M phases; the height of each bar indicates the average number of Ep-CAM molecules per cell within the total population.

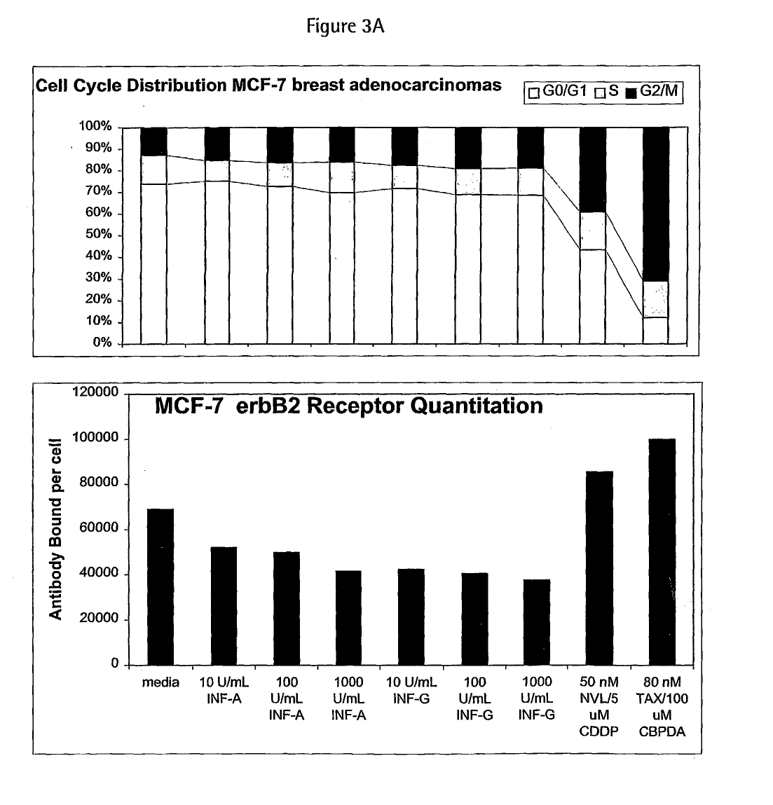

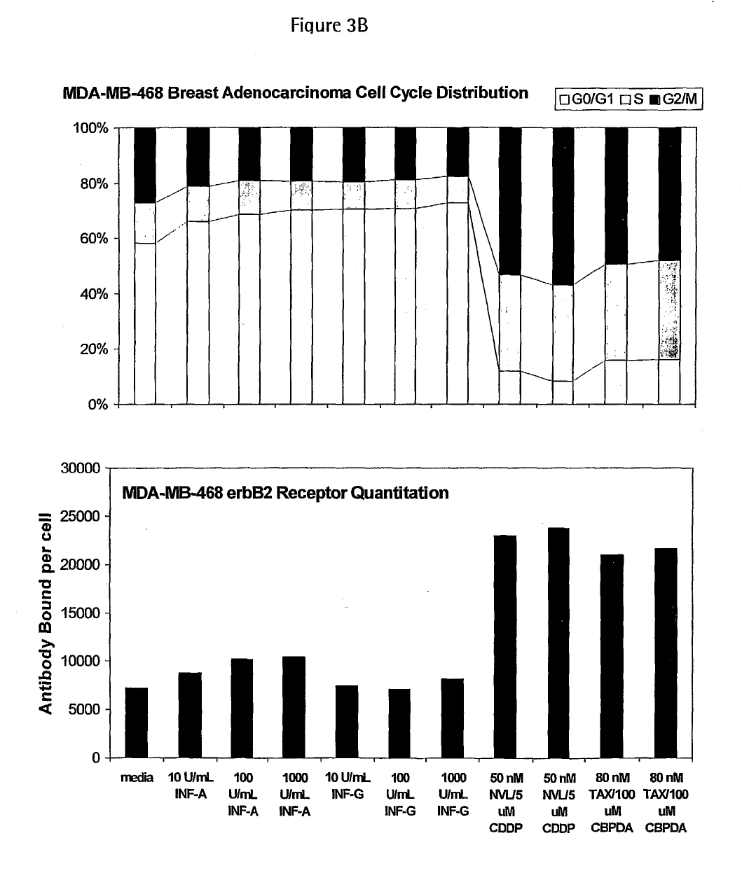

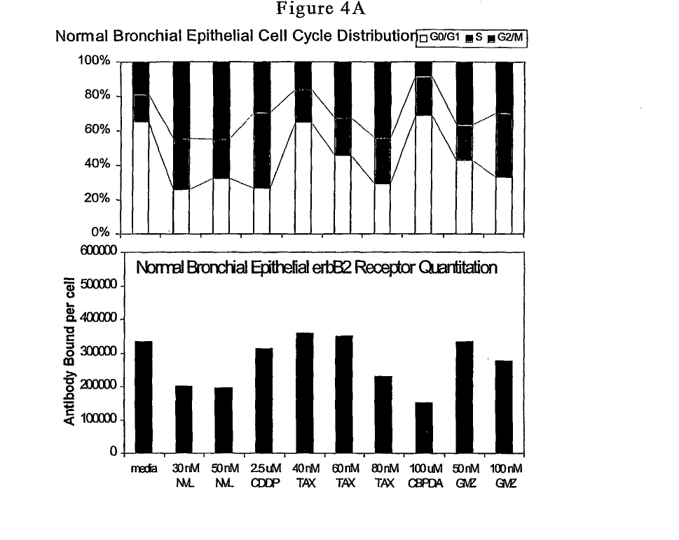

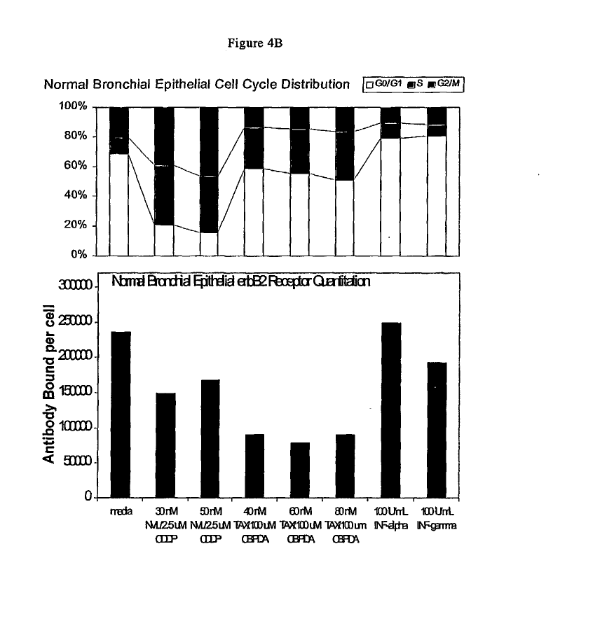

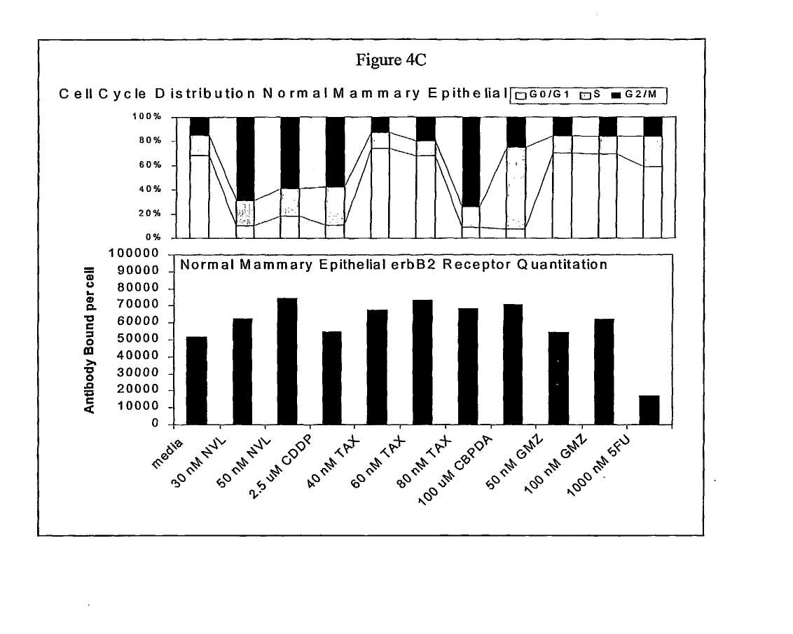

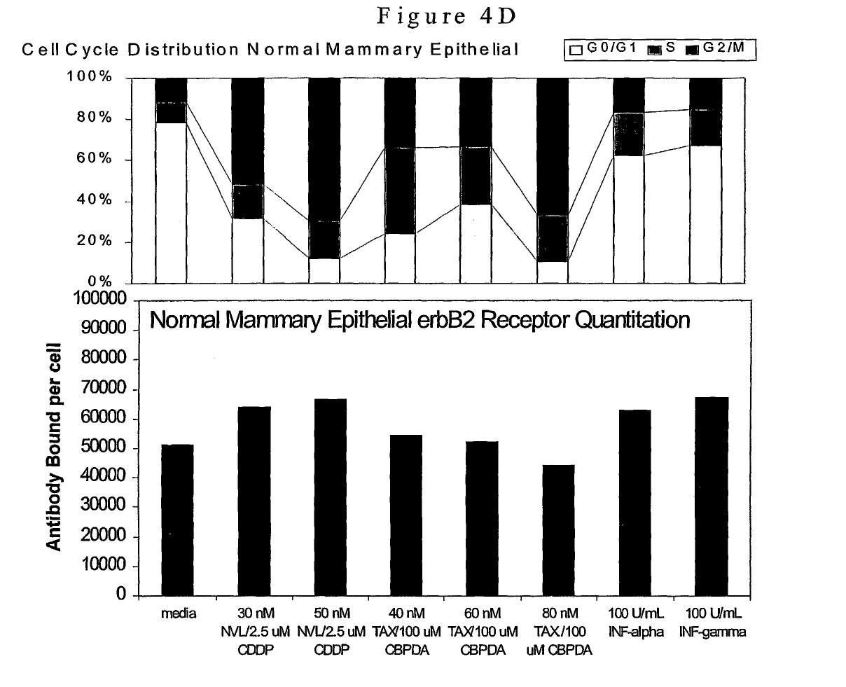

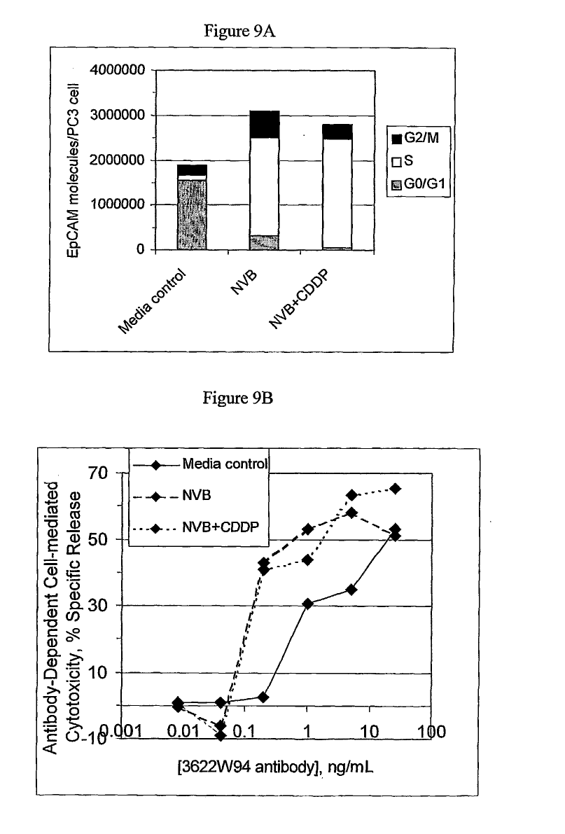

Increased Ep-CAM antigen presentation was observed on adenocarcinoma cells but not normal cells exposed to chemotherapeutic agents in culture. The cell surface presentation of Ep-CAM and cell cycle distribution was quantitated on a variety of adenocarcinoma cells as well as primary cultures of normal human cells. FIGS. 3 and 4 clearly demonstrated that the adenocarcinoma cells from colon (FIGS. 8A, 8B), lung (FIGS. 8C, 8D) and prostate (FIG. 9A) achieved cell cycle block much more effectively and expressed higher levels of Ep-CAM subsequent to exposure to cycle-specific drug combinations. The demonstrated increase in cell surface Ep-CAM presentation subsequent to drug treatment varied from 2-10-fold. The increase in Ep-CAM density was dose-dependent and correlated with the effectiveness of cycle block (data not shown). In contrast, the four normal cell lines did not achieve cycle block as effectively and did not show any increase in antigen presentation, which remained undetectable in 2 of the normal cell populations.

FIG. 9. Ep-CAM Antigen Expression and ADCC of Prostatic Adenocarcinoma in Culture. Biological Effectiveness in vitro as Measured by Antibody-dependent Cellular Cytotoxicity. PC-3 adenocarcinoma target cells were exposed to either Navelbine (30 nM) alone or Navelbine followed by cisplatin (2.5 μM) as described above and then harvested and seeded into 96-well plates for a ⁵¹Cr-release cytotoxicity assay. Target cells were cultured overnight with 100 μCi Na₂⁵¹CrO₄ (Amersham) in media, and then washed 3 times with RPMI-1640 containing 2 mM L-glutamine, 50 μg/mL gentamicin, and 10% heat-inactivated FBS. Fresh human peripheral blood mononuclear cells that had been allowed to adhere overnight, then added to drug-exposed ⁵¹Cr-loaded target cells at a 50:1 effector: target ratio. Spontaneous lysis wells (CPMₛₚₒₙₜₐₙₑₒᵤₛ) received media (no effectors) and total lysis wells (CPM_{Total}) received Triton-X-100. Cultures were incubated for 6 hours at 37° C./5% CO₂, then supernatants were harvested using Skatron filter frames (Skatron Instruments, Sterling Va.). Radioactivity was counted in a gamma counter and the percentage specific release, corrected for spontaneous lysis, was calculated. PC-3 prostatic adenocarcinoma cells in culture exposed to Navelbine followed by cisplatin were better targets for human ADCC activity in vitro than control cells. To determine if the increase in cell surface presentation of Ep-CAM would correlate to an increase in the biological effectiveness of a targeting antibody, PC-3 prostatic adenocarcinoma cells were pretreated with Navelbine alone or Navelbine followed by cisplatin and the in vitro lytic efficacy of the humanized antibody GW3622W94) was evaluated by ADCC. The results are shown in FIG. 8b. The ability of human peripheral blood ADCC effector cells to lyse tumor target cells coated with antibody was improved when the target cells had been pre-treated with Navelbine (30 nM) alone or in the presence of cisplatin (5 μM). In addition, low concentrations of antibody GW3622W94 (A 323/A3 humainsed antibody that binds Ep-CAM antigen and is constructed with murine CDRs within a human IgG1 framework. This reengineered humanised antibody is capable of interacting with Fc receptors on human effector cells for ADCC and of binding human complement C1q to initiate complement-mediated lysis. Following conjugation with 6,6′-bis{N,N,N″,N″-tetra(carboxymethyl)aminomethyl-4′-(3-bromoacetamido-4-methoxyphenyl-2,2′:6,2″-terpyridine, (TMT), this antibody became GW1208W95) (0.2 ng/mL=1/10 EC₅₀) were more effective at mediating ADCC of A549 (lung), DU145 (prostate) and H460 (lung) adenocarcinoma cells pre-treated with drugs (data not shown).

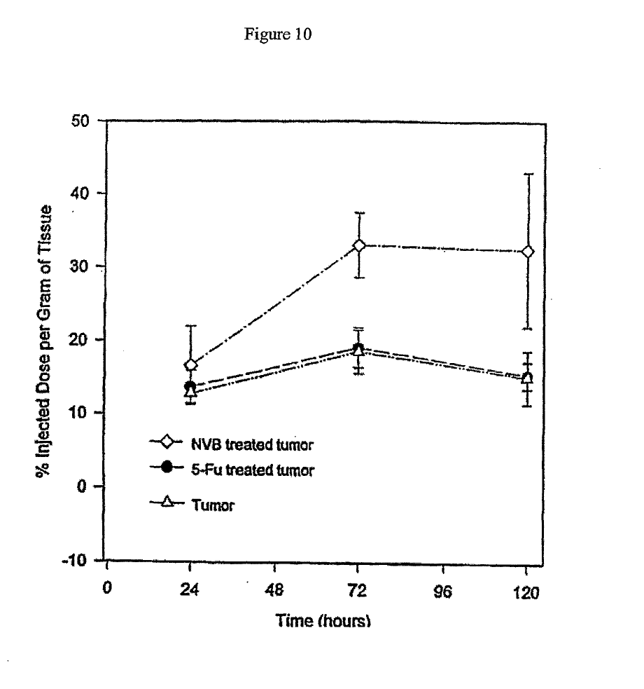

FIG. 10. Antibody targeting to Ep-CAM-positive xenografts was significantly improved by pre-treatment with Navelbine. Human colon adenocarcinoma (HT-29) tumors were initiated by subcutaneous implantation into female CD-1 nude mice (Charles River). When the tumors reached 200-300 mg, animals were divided into groups of five. Navelbine was injected intravenously at a dosage of 28 mg/kg in vehicle (5% dextrose in distilled water) on days 1 and 5. This dose of Navelbine was close to the LD₁₀ for this mouse strain and caused minimal tumor regression. A control group was dosed with 5-fluorouracil (5-FU) intraperitoneally at 20 mg/kg on days 1 and 5. On day 6, antibody GW1209W95 was labeled with lutetium-177 and injected intravenously via the lateral tail vein. Each mouse received a 200 μL injection containing 2.09 μCi ¹⁷⁷Lu-GW1209W95 (4.1 μg protein). Blood, spleen, liver, lung, kidney, femur, and tumor were harvested on days 1, 3 and 5 post-antibody-dose for direct gamma counting (Packard).

Because pretreatment of cells in culture with Navelbine caused an increase in the cell surface presentation of Ep-CAM, we investigated whether pretreatment with Navelbine would cause an increase in targeting to Ep-CAM expressing tumors (HT-29 colon adenocarcinoma) in CD-1 nude mice. FIG. 10 demonstrates that pretreatment with Navelbine resulted in a 2-fold increase in tumor targeting relative to vehicle- or 5-FU-treated animals.

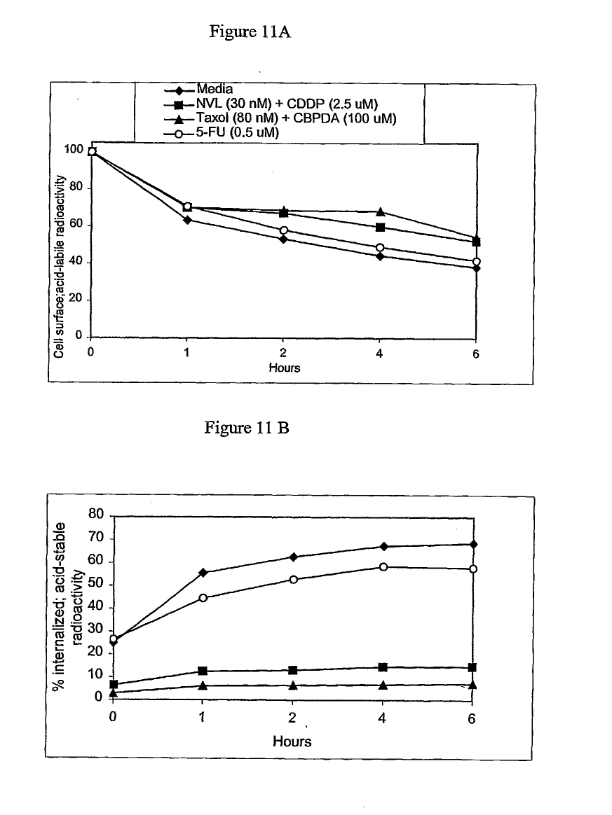

FIG. 11. Internalization of Ep-CAM antigen on HT-29 colon adenocarcinoma cells in culture was signficantly inhibited by pretreatment with chemotherapeutic agents. Internalization of Lutetium-177-labeled GW1208W95 (anti-Ep-CAM) by Human Colon Adenocarcinoma Cells. Colon adenocarcinoma cells (HT-29) were plated in 6-well plates and cultured. Subconfluent cells were exposed to cytotoxic drugs for up to 24 hours. Cells were washed and cultured for another 2-5 days. The plates were put on ice (0° C.) for minimum of 60 minutes and lutetium-177-labeled coupled to 6,6″-bis {N,N,N″,N″-tetra(carboxymethyl)aminomethyl)-4′-(3-bromoacetamido-4-methoxyphenyl-2,2′:6,2″-terpyridine) at 0.5-1.0 mCi/mg was added at a final concentration of 6.7 nM. Cells were incubated at 0° C. for 3.5 hr. Following incubation at 0° C., the cells were washed with 2.5 mL of ice-cold phosphate-buffered saline with human serum albumin (1%) (PBS-HSA). Then 2.5 mL pre-warmed media was added and the labeled cells were incubated at 37° C. At the indicated time points, cells were washed twice with 2.5 mL PBS-HSA. Cells were incubated for 5 minutes at room temperature with 2.5 mL 0.1 M NaCl in 0.05 M glycine (pH 2.8) to detemine labeled antibody bound to the target surface antigen. The incubation was repeated and counts pooled. Internalized counts were determined by solubilization of the cell layer with 1.0 mL of 1 N NaOH. Samples were counted using a Packard CliniGamma 5000. Data was normalized to cell count for each well. To demonstrate specificity of binding, unlabeled antibody GW1208W95 (130 nM) was added to labeled cells and then the cells were processed as above.

It has been well established that microtubules are involved in the internalization of cell surface antigens and it has been demonstrated previously that Ep-CAM does internalize (Kyriakos, R. J., Shih, L. B., Ong, G. L., Patel, K., Goldenberg, D. M. and Mattes, M. J. The Fate of Antibodies Bound to the Surface of Tumor Cells in Vitro” Cancer Research 52:835-842, 1992.). Since the agents evaluated in this study are microtubule targets, it seemed logical to evaluate the effect of these agents on the internalization of Ep-CAM. Colon adenocarcinoma cells (HT-29) in culture were pretreated with chemotherapeutic agents as described previously for the evaluation of cell cycle and cell surface antigen quantitation. Following treatment with either Navelbine (30 nM) alone or Navelbine followed by cisplatin (2.5 μM), cells were evaluated for antigen internalization using established literature protocols (Novak-Hofer, I., Amstutz, H. P., Morgenthaler J. and Schubiger, P. A. Internalization and Degradation of Monoclonal Antibody chCE7 by Human Neuroblastoma Cells. int. J. Cancer 57:427432, 1994.). Cells were pulse-labeled with ¹⁷⁷Lu-GW1208W95 (6.7 nM @ 0.5-1.0 mCi/mg protein) for a minimum of 60 minutes at 0° C. on ice to inhibit surface antigen internalization. After washing away of excess radiolabeled antibody fresh media was added, and the cells were incubated at 37° C. for time indicated. Surface-bound antibody was eluted with isotonic buffer and both acid-labile (surface-bound) and acid-stable (internalized) radioactivity were quantitated. FIG. 11A shows the disappearance of ¹⁷⁷Lu-GW1208W95 from the cell surface. Regardless of treatment, all cells appeared to have similar rates of radiolabelled antibody dissociation. Navelbine or Taxol treatment significantly inhibited internalization of the radiolabelled antibody (FIG. 11B). In contrast, cells that were treated with 5-FU or media had extensive internalization of the radiolabelled antibody. We demonstrated that binding of the radiolabelled antibody was specific for Ep-CAM by competing greater than 93% of the radioactivity using an 18-fold excess of unlabeled GW1208W95 (data not shown).

### EXAMPLE 1

### ErbB2/neu Presentation on Adenocarcinoma Cells Varied by Cell Cycle Phase

Populations of adenocarcinoma cells were evaluated for distribution in G₀/G₁, S and G₂/M phases of the cell cycle as well as for erbB2/neu presentation. Cells were dissociated from the culture plates using Versene (Gibco) and resuspended in calcium- and magnesium-free phosphate-buffered saline containing bovine serum albumin and sodium azide. Exactly 2×10⁵ cells were stained with R-Phycoerythrin-conjugated-anti-HER-2/neu murine IgG (Cat. 340552, Becton Dickinson) in buffer containing 100 μg/mL mouse IgG (Cat. 15381, Sigma). Cells were fixed with FACSLyse (Cat. 92-002, Becton Dickinson) followed by a short post-fix with ethanol at −20° C. Cellular DNA was stained with DAPI (Cat. D1306, Molecular Probes) in buffer containing RNase A (Sigma).

Collection and Analysis of Flow Cytometric Data

Sample data were collected on a FACStar^{PLUS}® flow cytometer (Becton Dickinson) equipped with a 488 nm argon ion laser in position 1 and a 350 nm argon ion laser in position 2. For each cell analyzed, data was collected on signal pulses from linear forward scatter height and width, linear area and width of DAPI fluorescence for DNA, and logarithmic fluorescence height of the HER-2/neu antibody probe. The resulting listmode files were processed using Winlist 3D© software (Verity Software House, Topsham, Me.). Displays of cell population data was used to discriminate doublets and aggregates revealed by forward scatter width and DAPI fluorescence width versus DAPI fluorescence area. The remaining cells were analyzed for cell cycle position by manual gating and HER-2/neu surface antigen density. Using the bead standard system described in Example 2, values of mean fluorescence intensity for HER-2/neu and other antigens were converted to values of “Antibodies Bound per Cell” (ABC) during analysis.

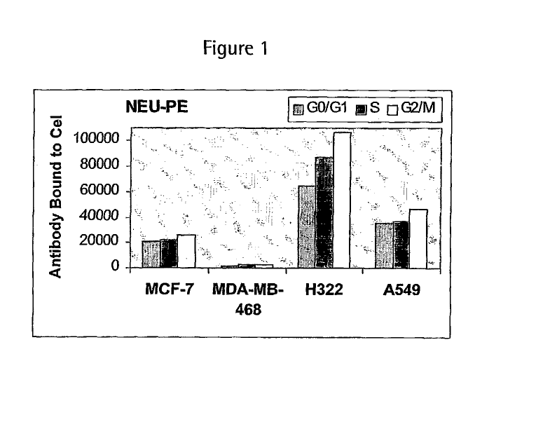

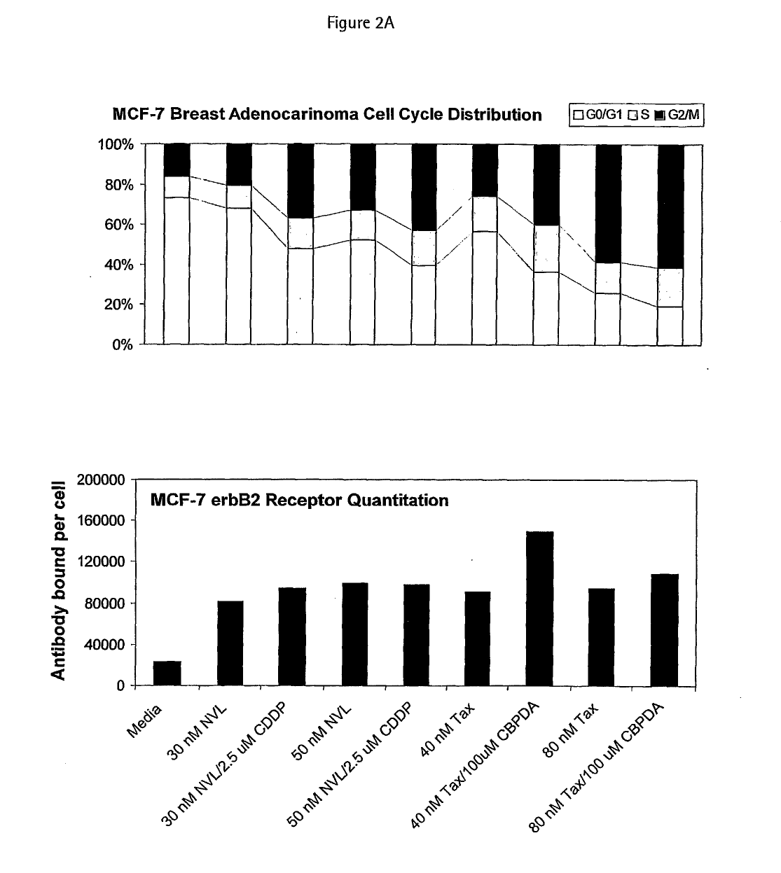

FIG. 1 shows that erbB2 is expressed across the cell cycle, but at higher density and greater homogeneity (data not shown) on cells in S and in G₂/M phases than in G₀/G₁. The examples include MCF-7 (breast), MDA-MB-468 (breast), H322 (lung) and A549 (lung) adenocarcinomas. This pattern of expression has been documented in all epithelial-derived tumors cells studied to date.

### EXAMPLE 2

### Increased Presentation of erbB2 Receptor on Adenocarcinoma Cells was Associated with Arrest of Cell Cycle Progression and Accumulation of Cells in S and G₂/M Phases.

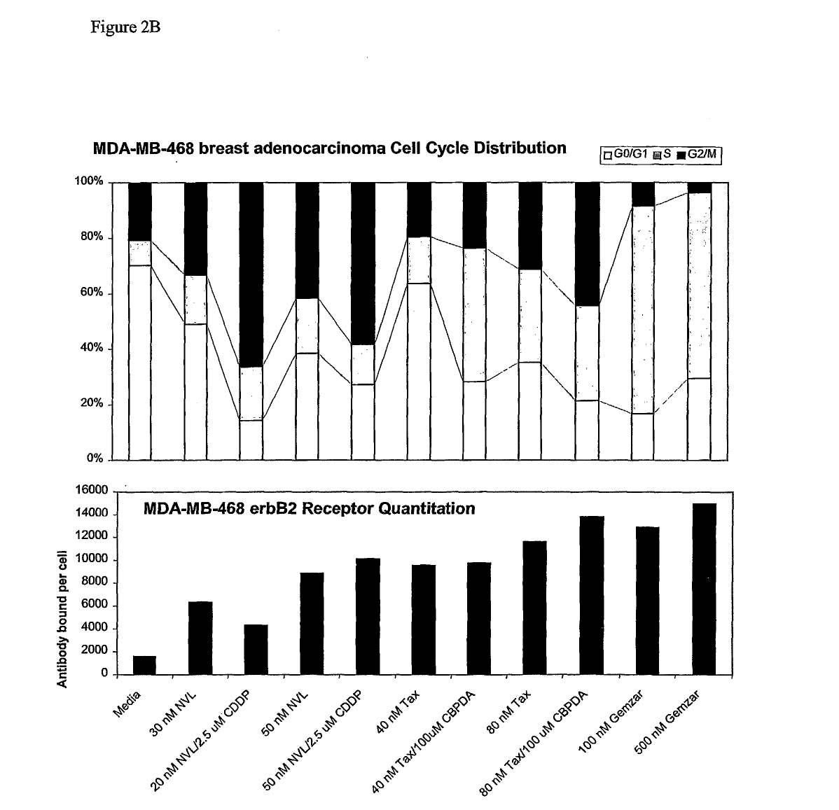

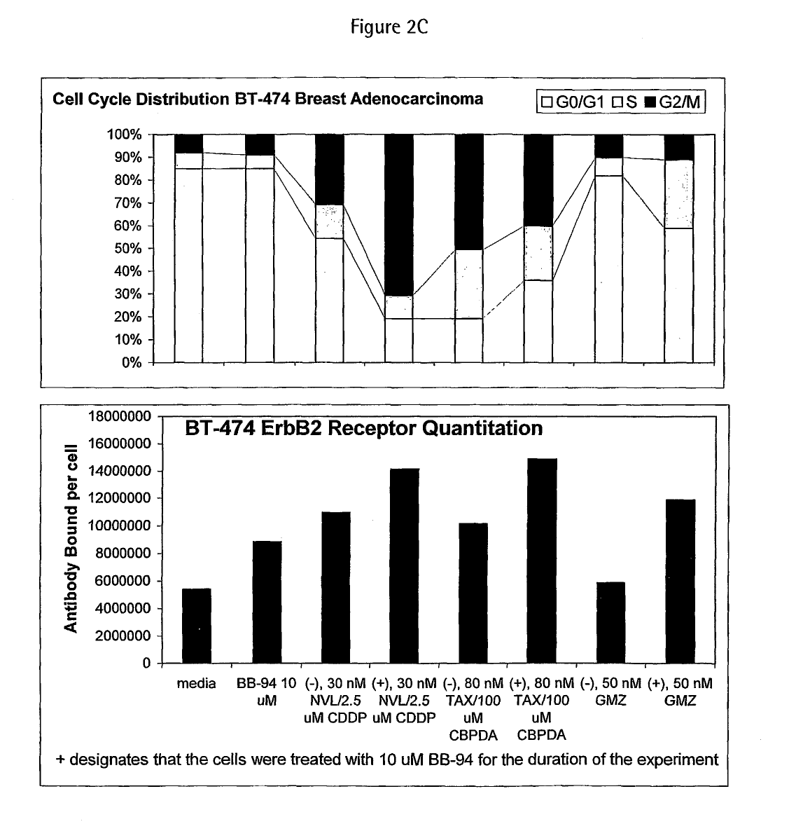

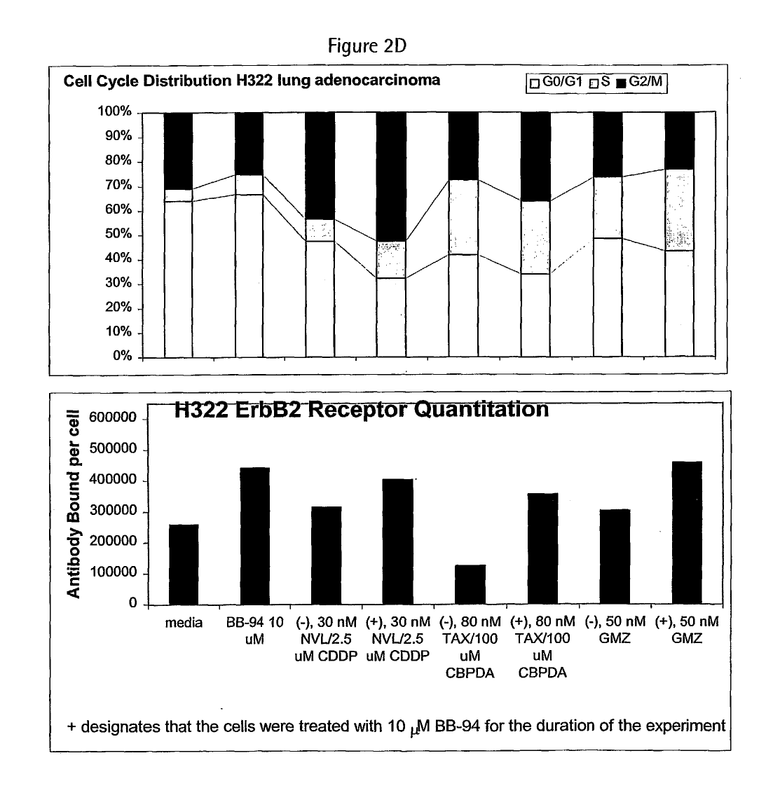

Adenocarcinoma cells were exposed to various drugs or combinations of drugs as indicated in FIGS. 2A-D. Subconfluent cells were exposed to vinorelbine (Navelbine® (NVL), Glaxo Wellcome, Inc., RTP, NC) or paclitaxel (Taxol (TAX), Bristol-Myers Squibb, Princeton, N.J.) for up to 24 hours, then washed and exposed to cisplatin (CDDP, Bristol Laboratories, Princeton N.J.) or carboplatin (Paraplatin® (CBPDA), Bristol Oncology, Princeton, N.J.). Cells were exposed to Gemzar (gemcitabine (GMZ), Lilly, Indianapolis, Ind.) for 24 hours. The high erbB2 expressing cell lines, BT-474 and NCI H322 (FIGS. 2C, 2D), were treated with the metalloprotease inhibitor BB-94 (10 μM) to prevent ectodomain shedding (Codony-Servat, J., Albanell, J., Lopez-Talvera, J. C., Arribas, J., and Baselga, J. “Cleavage of the HER-2 Ectodomain is a Pervanadate-activable Process That is Inhibited by the Tissue Inhibitor of Metalloproteases-1 in Breast Cancer Cells” Cancer Research 59, 1196-1201 (1999)). Following drug exposure, cells were washed and cultured for another 2-5 days prior to analysis for antigen presentation and cell cycle status, except for those treated with BB-94. Cells were dissociated from the culture plates using Versene (Gibco) and resuspended in calcium- and magnesium-free phosphatebuffered saline containing bovine serum albumin and sodium azide. Exactly 2×10⁵ cells were stained as described in Example 1. Antigen presentation was quantified against calibrated bead standards calibrated by the vendor for murine IgG binding capacity (Quantum Simply Cellular Bead, Cat. QSC-100, Sigma); calibration beads were stained with R-phycoerythrin-conjugated anti-HER-2/neu murine IgG. Plots of fluorescence intensity against bead IgG binding capacity were constructed, and molecules of IgG bound per cell was read from the fluorescence intensity of the stained cells.

As cells synthesise DNA and prepare to divide, the cell volume increases until mitosis occurs and thus, the relationship between cell cycle and cell size may translate to a greater surface area and possibly greater antigen expression, assuming equivalent surface density. Assuming that a cell in late M phase is twice the volume of a cell in G₀/G₁, the radius of the cell can be calculated as r³=volume/(4/3π), then the surface area can be calculated as Aₛ=4πr². Based on these assumptions, increases in antigen presentation of less than 1.6-fold may be due to cycle-related increased cell size, whereas increases greater than 1.6-fold are significant changes in the cell surface presentation (antigen density). A recent study (Pocsik, E., Mihalik, R., Penzes M., Loetscher, H., Gallati, H. and Aggarwal, B. Effect of Cell Cycle on the Regulation of the Cell Surface and Secreted Forms of Type I and Type II Human Tumor Necrosis Factor Receptors” J. of Cell. Biochem. 59:303-316, 1995.) in histiocytic lymphoma U-937 cells in culture supports our contention that the agents that block cells in various stages of the cell cycle do not significantly alter cell size beyond that attributable to cell cycle progression.

Cell cycle analysis demonstrated that approximately 30% of media control cells were in S and G₂/M phases combined with both MCF-7 (FIG. 2A) and MDA-MB-468 (FIG. 2B) breast adenocarcinoma cells. Effective concentrations of Navelbine or Taxol alone or in combination with cisplatin or carboplatin, respectively, increased cells blocked in S and G₂/M to greater than 70%. In addition, these agents alone or in combination caused significant increases in cell surface erbB2 presentation compared with untreated controls. The increase in erbB2 presentation was dose dependent and correlated with percentage of cells in S and G₂/M phases.

The high erbB2 receptor-presenting cell lines, NCI H322 lung and BT-474 breast adenocarcinomas, shed the extracellular domain of the receptor (Codony-Servat, J., Albanell, J., Lopez-Talvera, J. C., Arribas, J., and Baselga, J. “Cleavage of the HER-2 Ectodomain is a Pervanadate-activable Process That is Inhibited by the Tissue Inhibitor of Metalloproteases-1 in Breast Cancer Cells” Cancer Research 59, 1196-1201 (1999)) and prevented accurate quantitation of receptor presentation. Therefore, the cells were treated with a broad-spectrum metalloprotease inhibitor, BB-94, that blocked erbB2 extracellular domain shedding and facilitated quantitation of erbB2 receptor presentation. Cell cycle analysis demonstrated that approximately 15% and 35% of media control cells were in S and G₂/M phases combined with BT-474 (FIG. 2C) and H322 (FIG. 2D), respectively. While treatment with BB-94 alone did not affect cell cycle distribution, both cell lines displayed an increase in erbB2 presentation. Effective concentrations of Navelbine and Taxol in combination with cisplatin or carboplatin, respectively, and Gemzar increased the cells blocked in S and G₂/M to 40-70% compared to untreated controls. Furthermore, these agents caused significant increases in cell surface erbB2 presentation. In all cases, the highest increases were seen in the presence of BB-94.

### EXAMPLE 3

### Interferon Treatment had no Effect on Cell Cycle Distribution or erbB2 Receptor Presentation

Adenocarcinoma cells were exposed to various drugs or combinations of drugs as indicated in FIGS. 3A and B. Subconfluent cells were exposed to vinorelbine (Navelbine® (NVL), Glaxo Wellcome, Inc., RTP, NC) or paclitaxel (Taxol (TAX), Bristol-Myers Squibb, Princeton, N.J.) for up to 24 hours, then washed and exposed to cisplatin (CDDP, Bristol Laboratories, Princeton N.J.) or carboplatin (Paraplatinm (CBPDA), Bristol Oncology, Princeton, N.J.). Cells were exposed to interferons continuously for 2-5 days. Cells were dissociated from the culture plates using Versene (Gibco) and resuspended in calcium- and magnesium-free phosphatebuffered saline containing bovine serum albumin and sodium azide. Exactly 2×10⁵ cells were stained as described in Example 1, and antigen expression was quantified as described in Example 2.

Cell cycle analysis demonstrated that MCF-7 (FIG. 3A) and MDA-MB-468 (FIG. 3B) breast adenocarcinomas exposed to increasing concentrations of INF-α or INF-γ were not significantly different from media control cells. In addition, cell surface presentation of erbB2 receptor was not significantly increased in cells exposed to these agents. These results contrasted with exposure to Navelbine plus cisplatin or Taxol plus carboplatin, that resulted in accumulation of cells in S or G₂/M phases and significant increases in erbB2 receptor presentation.

### EXAMPLE 4

### Increased erbB2 Receptor Presentation was not Observed on Normal Cells Exposed to Cytotoxic Agents in Vitro

Normal human epithelial cells from lung (NHBE, Clonetics®) and mammary (HMEC, Clonetics®) were exposed to various drugs or combinations of drugs as indicated in FIGS. 4A-D. Subconfluent cells were exposed to vinorelbine (Navelbine (NVL), Glaxo Wellcome, Inc., RTP, NC) or paclitaxel (Taxol (TAX), Bristol-Myers Squibb, Princeton, N.J.) for up to 24 hours, then washed and exposed to cisplatin (CDDP, Bristol Laboratories, Princeton N.J.) or carboplatin (Paraplatin® (CBPDA), Bristol Oncology, Princeton, N.J.). Cells were exposed to Gemzar (gemcitabine (GMZ), Lilly, Indianapolis, Ind.) or 5FU (Adrucil®, Pharmacia & Upjohn) for 24 hours. Cells were exposed to interferons continuously for 2-5 days. Following drug exposure, cells were washed and cultured for another 2-5 days prior to analysis for antigen presentation and cell cycle status, except for those treated with interferons. Cells were dissociated from the culture plates using a collagenase cocktail (1:1:1,Types I, II, and IV, 0.1% (wt/vol), Gibco) in calcium- and magnesium-free phosphate-buffered saline. Cells were stained and cytometric data was collected as described in Example 1, and antigen expression was quantified as described in Example 2.

Cell cycle analysis demonstrated that approximately 30% of media control cells were in S and G₂/M phases combined with both NHBE (bronchial, FIGS. 4A, 4B) and IMEC (mammary, FIGS. 4C, 4D) normal epithelial cells. Effective concentrations of Navelbine or Taxol alone or in combination with cisplatin or carboplatin, respectively, increased cells blocked in S and G₂/M to greater than 70%. However, these agents alone or in combination caused no significant increases in cell surface erbB2 presentation compared with untreated controls.

### EXAMPLE 5

### Increases in erbB2 Receptor Presentation Caused by Navelbine and Taxol are not a Result of Increased Gene Expression

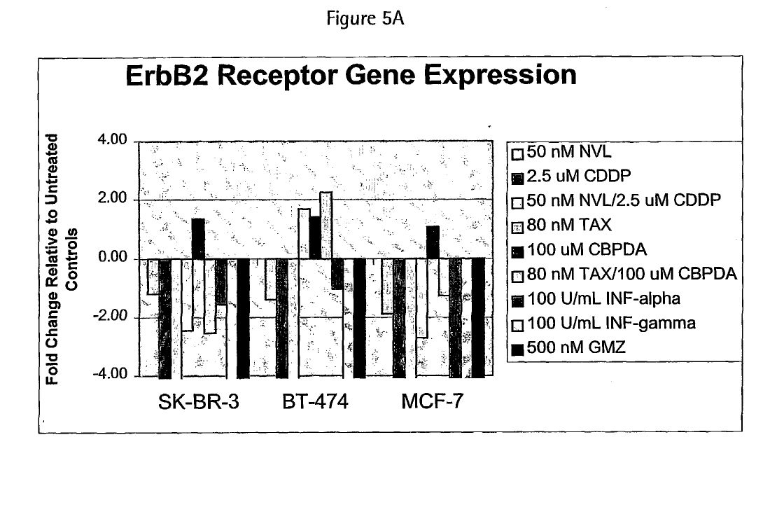

Adenocarcinoma cells were exposed to various drugs or combinations of drugs as indicated in FIG. 5. Subconfluent cells were exposed to vinorelbine (Navelbine® (NVL), Glaxo Wellcome, Inc., RTP, NC) or paclitaxel (Taxol (TAX), Bristol-Myers Squibb, Princeton, N.J.) for up to 24 hours, then washed and exposed to cisplatin (CDDP, Bristol Laboratories, Princeton N.J.) or carboplatin (Paraplatin® (CBPDA), Bristol Oncology, Princeton, N.J.). Cells were exposed to Gemzar (gemcitabine (GMZ), Lilly, Indianapolis, Ind.) for 24 hours. The drugs and concentrations used in this study were known to cause cell cycle arrest from examples cited previously. Cells were exposed to interferons continuously for 2-5 days. Following drug exposure, cells were washed and cultured for another 2-5 days, except for those treated with interferons. Cells were washed and stored in lysis buffer prior to mRNA extraction.

Preparation of RNA from Cell Lines Treated with Chemotherapeutics

RNA was isolated by the ABI 6700 (Applied Biosystems) according to manufacturer's protocols from five 96-well plates containing cell lines that were exposed to either media only or various chemotherapeutics. The amount of RNA isolated was determined by real-time PCR analysis of the 18 s rRNA. Briefly, 5 μg of a 1:100 fold dilution of total RNA was added to a 96-well plate that contained a 20 μl cocktail mixture of 5.5 mM MgCl₂, 1×Buffer A, 300 μM dNTP, 10 U RNase inhibitor, 12.5 U MuLV reverse transcriptase, 1.25 U Amplitaq Gold (Applied Biosystems, Foster City, Calif.), 40 nM of forward primer (5′CGCCGCTAGAGGTGAAATTCT 3′), 20 nM reverse primer (5′CATTCTTGGCAAATGCTTTCG 3′) and 50 nM of Probe (5′ Joe-6-carboxy-4,5 dichloro-2,′,7′-tetrachlorofluorescein-ACCGGCGCAAGACGGACCAGA-TAMRA-6-carboxy-N,N,N′N′-tetramethylrhodamine 3′). The probe is covalently bound to a 5′ reporter dye and a 3′ quencher dye. Water is added to the reaction to give a final volume of 25 μl and the mixture is placed in an ABI Prism 7700 Sequence Detection thermocycler (Applied Biosystems). The reaction is heated to 48° C. for 30 minutes, then 95° C., 10 minutes followed by 40 cycles at 95° C., 15 seconds and 60° C. for 1 minute. The amount of 18 s rRNA in each sample was determined by the amount of fluorescence (the number of molecules) at the cycle threshold (Ct) and calculated against a standard curve (Strum, J. C., Carrick. K. M., Stuart, J. S. and Martensen, S. A. Tissue Expression Profiling using Real-time PCR. Current Protocols in Pharmacology (In Press) (2001); Bustin, S. A. Absolute quantification of mRNA using real-time reverse transcription polymerase chain reaction. J. Mol. Endo. 25, 169-193 (2000).).

ErbB2 Gene Expression Analysis of Treated RNA

Five microliters of RNA was transferred to a replicate 96-well plate containing a RT-PCR reaction cocktail, as outlined above. For ErbB2, 300 nM of forward primer (5′GGATGTGCGGCTCGTACAC 3′), 300 nM of reverse primer (5′GTAATTTTGACATGGTTGGGACTCT 3′) and 150 nM of probe (5′ FAM (6-carboxyfluorescein)-ACTTGGCCGCTCGGAACGTGC-TAMRA 3′) was added to the cocktail mixture. Real-time PCR analysis of the RNA for ErbB2 expression was carried out using the standard laboratory protocols as outlined previously (Strum, J. C., Carrick. K. M., Stuart, J. S. and Martensen, S. A. Tissue Expression Profiling using Real-time PCR. Current Protocols in Pharmacology (In Press) (2001)). ErbB2 gene expression was also measured in the absence of reverse transcriptase to determine the amount of genomic DNA contaminants present. All samples contained little to no genomic DNA.

Calculation of the Amount of Gene Expression

The amount of gene expression for each cell line was determined by comparing the gene expression of the treatment group to the control group. The ΔCt value was determined by subtracting the Ct value of the treatment group from the Ct value of the control group. The fold equation (2^{ΔCt}) for each treatment group was determined and the significance difference reported to be 2 fold or greater.

Quantitation of erbB2 receptor mRNA demonstrated that exposure of SK-BR-3, MCF-7 and BT-474 breast adenocarcinomas (FIG. 5A) and MDA-MB-468 breast adenocarcinoma (FIG. 5B) cells in culture to agents that had been shown previously to arrest cells in S and G₂/M phases of the cell cycle did not significantly increase erbB2 receptor gene expression. The mRNA for each treatment was normalised to 18 s values to account for cell number variability due to drug exposure. In most cases, treatment caused a minimal change in erB2 gene expression (within a 2-fold change relative to untreated controls) or a decrease in erbB2 gene expression. Cisplatin (CDDP), Gemzar (GMZ) and INF-γ were toxic to SK-BR-3, MCF-7 and BT-474 breast adenocarcinoma cell lines (FIG. 5A) based on the 18 s values, resulting in low ratios. It appears that the increases in erbB2 receptor presentation that we have seen following exposure of cell lines to agents that block cell cycle arrest in G₂/M are not due to increased expression of erbB2 receptor gene.

### EXAMPLE 6

### General Protocol for the Quantitation of Cell Surface Targets Following Pre-Treatment with G₂/M Agents

Cells in culture that present a cell surface target(s) of interest are identified and exposed to various drugs or combinations of drugs as indicated. Subconfluent cells were exposed to vinorelbine (Navelbine® (NVL), Glaxo Wellcome, Inc., RTP, NC) or paclitaxel (Taxol (TAX), Bristol-Myers Squibb, Princeton, N.J.) for up to 24 hours, then washed and exposed to cisplatin (CDDP, Bristol Laboratories, Princeton N.J.) or carboplatin (Paraplatin® (CBPDA), Bristol Oncology, Princeton, N.J.). Cells were exposed to Gemzar (gemcitabine (GMZ), Lilly, Indianapolis, Ind.) for 24 hours. Cells were exposed to interferons continuously for 2-5 days. Agent concentration and duration of exposure are optimised for maximal cell cycle block in G₂/M and minimal cell death. Cells were dissociated from the culture plates while maintaining the integrity of the cell surface target using Versene (Gibco), trypsin (Gibco), or collagenase (Gibco) and resuspended in calcium- and magnesium-free phosphatebuffered saline containing bovine serum albumin and sodium azide. Exactly 2×10⁵ cells were stained with a fluorescent-conjugated antibody(ies) that binds with high affinity to the cell surface target(s) of interest in buffer containing 100 μg/mL mouse IgG (Cat. 15381, Sigma). Cells were fixed with FACSLyse (Cat. 92-002, Becton Dickinson) followed by a short post-fix with ethanol at −20° C. Cellular DNA was stained with Propidium Iodide (Molecular Probes) or DAPI (Molecular Probes) in buffer containing RNase A (Sigma).

Collection and Analysis of Flow Cytometric Data

Sample data were collected on a FACStar^{PLUS}® flow cytometer (Becton Dickinson). For each cell analysed, data were collected on signal pulses from linear forward scatter height and width, linear area and width of DAPI fluorescence for DNA, and logarithmic fluorescence pulse height of the cell surface target(s) of interest antibody probe. The resulting listmode files were processed using Winlist 3D® software (Verity Software House, Topsham, Me.). Displays of cell population data were used to discriminate doublets and aggregates revealed by forward scatter width and DAPI fluorescence width versus DAPI fluorescence area. The remaining cells were analysed for surface antigen density and for cell cycle position by manual gating. Antigen presentation was quantified against bead standards calibrated by the vendor for murine IgG binding capacity (Quantum Simply Cellular Bead, Cat. QSC-100, Sigma); calibration beads were stained with R-phycoerythrin-conjugated anti-HER-2/neu murine IgG. Plots of fluorescence intensity against bead IgG binding capacity were constructed, and molecules of IgG bound per cell was read from the fluorescence intensity of the stained cells.

### EXAMPLE 7

### A Generalised Protocol for Determining Biological Data Tumor Studies

Target cells were cultured in RPMI 1640+10% Fetal bovine serum, Sodium pyruvate and L-Glutamine at 37° in a 95/5% air/CO₂ atmosphere. Cells were harvested following trypsin digestion and brought to a density of 2×10⁶ cells/200 μl in PBS. Tumors were initiated by injection of the cell suspension subcutaneously in the axillary region.

Tumor Studies: Measurements

For the xenograft models used here solid tumors were measured by electronic caliper measurement through the skin, measurements were typically made twice weekly. In the examples presented, tumors were monitored beyond the duration of therapy

Tumor Studies: Formulation and Administration

Drugs were administered by P.O. or I.V. routes. The G₂/M agent was formulated in aqueous 0.5% hydroxypropyl methylcellulose, 0.1% Tween 80 and administered as a suspension twice daily for 21 days as indicated in the respective figures. Taxol® (Bristol Myers Squibb Co.) was purchased preformulated in Cremophor-EL saline and diluted into saline to a final Cremophor-EL concentration of 5 or 10% Cremophor-EL for 10 or 20 mg/kg Taxol therapy respectively. Taxol was administered I.V., once a day, for 5 days (days 1-5) as indicated in the respective figures. Carboplatin (Sigma) was formulated in saline and was administered I.V., once a day, for two 5 day periods.These studies were performed under IACUC # 468.

1. A pharmaceutical combination comprising a G₂/M agent and a therapeutic agent whose therapeutic effectiveness is dependent, at least in part, on the presence of an internalising cell surface structure on the target cell.

2. A combination of claim 1 together with instructions for administration to a mammalian patient.

3. A combination of claim 1 or 2 wherein the G₂/M agent is selected from the group consisting of;
Vinorelbine tartrate, cisplatin, carboplatin, paclitaxel, doxorubicin, 5FU, docetaxel, vinblastine, vincristine, cyclophosphamide, apigenin, genistein, cycloxazoline.

4. A combination of any preceding claim wherein the structure is a protein or modified protein (e.g. glycoprotein), seven transmembrane receptor or antigen.

5. A combination of claim 4 wherein the structure is a tyrosine kinase or serine/threonine kinase.

6. A combination of claim 5 wherein the structure is c-erB2, c-erbB3, c-erbB4, c-fms, folate, β-integrins, VEGFR-2, EDG-1, IGF-1.

7. A combination of any preceding claim wherein the therapeutic agent is an antibody or a small molecule therapeutic.

8. A combination of claim 7 wherein the antibody is a chimaeric or humanised antibody.

9. A combination of claim 7 or 8 wherein the antibody is conjugated to a toxin or radionuclide.

10. A method for identifying a G₂/M agent which method comprises the steps of:
(a) providing a candidate agent;
(b) contacting said agent with preferably a mammalian cell, preferably a human cell, even more preferably a cancerous or pre-cancerous human cell;
(c) determining whether the density (i.e. number) of a cell surface structure which structure is indicative of the G₂/M stage of the cell cycle is increased;
(d) selecting said agent which causes said increase of step (c);
(e) Optionally synthesising and/or purifying said agent of step (d).

11. A method of treating a mammalian patient (afflicted with e.g. a disease of cell cycle regulation) in clinical need comprising the steps of;
(a) screening a candidate agent for the ability to increase the cell surface density of a G₂/M internalising cell surface structure of a cell;
(b) selecting an agent which causes an increase in said cell surface density; (c) Simultaneously treating said patient with a therapeutically effective amount of; said agent of step (b) and a therapeutic agent which specifically binds to a G₂/M internalising cell surface structure, preferably said structure of step (a).

12. A method for the treatment of a mammalian patient afflicted with a disease or disorder such as cancer, which method comprises the steps of;
(a) providing a G₂/M agent preferably by determining whether said agent has the ability to increase the cell surface density of a cell surface structure that is indicative of the G₂/M stage of the cell cycle, e.g. a G₂/M internalising cell surface structure;
(b) providing a therapeutic agent which specifically binds to or otherwise interacts with a G₂/M internalising cell surface structure, preferably said structure of step (a) optionally by determining whether a candidate therapeutic agent binds to (e.g. specifically binds to) an internalising G₂/M cell surface structure;
(c) simultaneously treating said patient with a therapeutically effective amount of said G₂/M agent of step (a) and said therapeutic agent of step (b).

13. A method of treating a mammalian patient, preferably human, in clinical need thereof which method comprises the step of simultaneously treating said patient with a G₂/M agent and a therapeutic agent whose therapeutic effectiveness depends at least in part on the expression on the cell surface of the patient cell a cell surface structure that internalises as the cell progresses through its cell cycle.

14. A method of treating a mammalian patient, preferably human, in clinical need thereof which method comprises the step of simultaneously treating said patient with a G₂/M agent and a therapeutic agent wherein treatment with said G₂/M agent blocks or retards progression of the cell cycle in said target cell at G₂ and/or M thereby increasing the density (i.e. number) of a cell surface structure (particularly an internalising cell surface structure) which is targeted by (e.g. specifically bound by) said therapeutic agent.

15. A method of treating a mammalian patient in clinical need thereof, which method comprises;
(a) administrating a G₂/M agent to increase the density of an antigen or receptor, particularly an internalising antigen or receptor on the target cell of the patient;
(b) administrating a therapeutic agent such as an antibody which specifically binds the antigen or receptor on said target cell of step (a) having increased antigen/receptor density;
(c) optionally reducing or removing the blocking effect of the G₂/M agent thereby permitting the target cell of step (b) to progress through the cell cycle and internalise the agent of step (b).

16. A method according to any one of claims 11 to 15 wherein the patient is afflicted with a cancer selected from the group consisting of; colorectal cancer, breast cancer, gastric cancer, prostate cancer, non-small cell lung cancer, lymphoma (e.g. Non-Hodgkins lymphoma), sarcoma, leukaemia.
